(19) United States
(12) Patent Application Publication
(10) Pub. No.: US 2020/0071648 A1
(43) Pub. Date: Mar. 5, 2020

(54) INTEGRATED MICROELECTRODES AND METHODS FOR PRODUCING THE SAME

(71) Applicant: THE ADMINISTRATORS OF THE TULANE EDUCATIONAL FUND, New Orleans, LA (US)

(72) Inventors: Michael James Moore, New Orleans, LA (US); Clayton B. Ford, Dearborn, MI (US); Xin Kai Yang, Williamsville, NY (US); Mohamed Aly Saad Aly, Scarborough (CA)

(21) Appl. No.: 16/604,974

(22) PCT Filed: Apr. 12, 2018
(86) PCT No.: PCT/US2018/027386

§ 371 Date: Oct. 11, 2019

Related U.S. Application Data
(60) Provisional application No. 62/484,500, filed on Apr. 12, 2017.

(51) Int. Cl. C12M 1/12; C12M 1/42; G01N 33/483; C12M 1/22; A61N 1/08; A61N 1/18

(52) U.S. Cl. CPC C12M 25/04; C12M 35/02; G01N 33/4836; A61N 1/04; A61N 1/08; A61N 1/18; C12M 23/10

(57) ABSTRACT

The disclosure relates to a tissue culture device and components of a system used to grow, maintain and measure recording from cells. In some embodiments, the tissue culture device is an insert with a surface onto which cells may be plated and grown. Electrodes on or near the surface of the cells can be used to measure electrophysiological data when current is applied to the system.

### CROSS-REFERENCE TO RELATED APPLICATIONS

This application is an international application designating the United States of America and filed under 35 U.S.C. §120, which claims priority to U.S. Provisional Application No. 62/484,500, filed on Apr. 12, 2017, which is herein incorporated by reference in its entirety.

### STATEMENT REGARDING FEDERALLY SPONSORED RESEARCH

This invention was made with government support under GA-2016-238 awarded by CASIS under a Cooperative Agreement with NASA. The government has certain rights in the invention.

### FIELD OF INVENTION

The present disclosure generally relates to custom inserts and multielectrode arrays (MEAs) for in vitro electrophysiological measurements from cells, and methods of producing and using the devices.

### BACKGROUND

Globally, neurological disease constitutes a signification portion of the global burden of disease [1]. Despite this, major neurological diseases such as multiple sclerosis (MS), which affects approximately 2 5 million people globally [1], remain poorly understood. Other diseases, such as diabetic neuropathy—a loss of nervous function due to glucose toxicity—are better understood, yet still impact many people with irreversible damage [2]: 75 thousand people in the United States in 2007 [3].

In order to develop treatments for such prevalent conditions, an effective model of the nervous system is necessary. Frequently, in vivo models are utilized for this purpose, such as the rat sciatic nerve model [4],[5]. Conducting research using an in vivo model however is very costly, and requires much manual effort. To conduct the large-scale screenings of compounds necessary for pharmaceutical drug discovery and testing, an in vitro model is much more attractive due to lower cost and the possibility of automation.

Typical in vitro models of nervous tissue are cultured on a surface, which results in robust growth, yet does not replicate in vivo conditions or morphology well, mainly due to a lack of three-dimensional (3D) extracellular matrix [6],[7]. To create an in vitro model of peripheral nervous tissue that more accurately represents in vivo conditions, our lab has developed a photolithographic method to polymerize polyethylene glycol diacrylate (PEG) hydrogels into replicable 3D structures, which are complemented with a second hydrogel capable of supporting neural growth[8]. Our lab has demonstrated that these dual hydrogel constructs are capable of supporting robust three dimensional neural growth that resembles in vivo nervous tissue[6].

The physiology of a nervous tissue can be analyzed through its electrophysiological response to stimuli, and characteristics of that response change when the tissue is subjected to pharmacological or pathological effects [9],[10]. This makes electrophysiology a useful tool for evaluating the effects of drugs or disease states on neural tissue, giving a snapshot of its functionality. These functional changes can be identified using field potential recording electrodes [11],[12], which have successfully been applied to electrophysiological evaluation of our dual-hydrogel neurite constructs [13]. However, such evaluation is tedious, as the proper placement of the stimulation and recording electrodes is an arduous and time-consuming task.

A major alternative to the usage of probes or electrode arrays for electrophysiology that is gaining in popularity is optogenetics, coupled with voltage-sensitive dyes that allow for the electrophysiological stimulation and recording using light exclusively, removing the necessity for direct contact [14]. However, such methods require complex microscope setups, require genetic modifications to the subject tissue, and render existing electrophysiological equipment moot.

### SUMMARY OF EMBODIMENTS

To navigate around the issues present in using field potential recordings or optogenetics to conduct electrophysiological analysis of our dual hydrogel neurite constructs, a platform was devised, including custom inserts and multielectrode arrays (MEAs) on which neurite constructs were formed and grown, and a custom rig to allow for rapidly interfacing the MEAs with electrophysiological test equipment. This platform was shown to be sufficient to view neurite responses to applied stimuli, and offers promise for rapid and automated use of our dual hydrogel model to perform large-scale pharmaceutical or pathological research.

The present disclosure relates to an insert comprising: (i) a permeable solid support comprising a top surface and a bottom surface; the top surface horizontal or substantially horizontal relative to a surface onto which the bottom surface of the insert lies, the top surface divided into an inner portion and an outer portion by one or a plurality of protrusions extending vertically from the top surface; wherein at least one region of the inner portion of the top surface defines the bottom face of a vessel and wherein the one or plurality of protrusions define one or more contiguous sidewalls of the vessel; (ii) one or plurality of electrodes physically attached to the top surface of the permeable solid support and positioned within the vessel; and (iii) one or plurality of contact pads, positioned on top of the at least one region of the outer portion of the top surface.

In some embodiments, the one or plurality of electrodes are planar in shape with a top and a bottom surface, the bottom surface of the one or plurality of electrodes positioned adjacent or substantially adjacent to the bottom face of the vessel.

In some embodiments, the flexular modulus of the permeable solid support is from about 0.2 to about 20 Gigapascals (GP).

In some embodiments, the insert comprises a first electrode and a second electrode, the first and second electrodes aligned in parallel in respect to a longitudinal axis but positioned proximate to opposite facing surface of the sidewalls.

In some embodiments, the insert comprises a first protrusion that is circular or substantially circular physically attached to the top surface on its edge defining the sidewalls of the vessel with a height from about 1 millimeter to about 15 millimeters above the top surface. In some embodiments, the permeable solid support is circular or substantially circular, semi-circular in shape and the one or plurality of electrodes are flat or substantially flat and are positioned adjacent to the top surface such that a longitudinal axis is parallel to the top surface of the permeable solid support; and the insert comprises at least four contact pads positioned around the outer portion of the permeable solid support.

In some embodiments, the insert further comprises a circular or semi-circular ring affixed to the permeable solid support, such that the permeable solid support and the ring define a cylindrical or substantially cylindrical vessel with a height of from about 0.5 to about 10 millimeters.

In some embodiments, the insert further comprises a hydrogel matrix layer positioned across the bottom face of the vessel. In some embodiments, at least one portion of the electrode is positioned below a top surface of the hydrogel matrix layer or protruding just above a top layer of the hydrogel matrix layer. In some embodiments, the hydrogel matrix forms a layer with a height from about 5 to about 500 microns. In some embodiments, the hydrogel matrix comprises a cavity with a depth from about 5 to about 500 microns. In some embodiments, the bottom region of the cavity has a surface area of from about 500 to about 5000 square microns.

In some embodiments, the insert further comprises one or a plurality of isolated Schwann cells; and one or a plurality of dorsal root ganglion (DRG) or DRG fragments.

In some embodiments, a first hydrogel matrix is layered across the top surface and comprises at least a first cavity, the cavity comprising a contiguous side region and a bottom region; wherein at least one portion of the electrode is positioned below the bottom region or protruding minimally above the bottom region; and wherein the one or plurality of isolated Schwann cells and/or the one or plurality of DRG or DRG fragments is positioned on top of the bottom region of the cavity such that the Schwann cells, DRG or DRG fragments are positioned above or are in contact with the one or plurality of electrodes.

In some embodiments, the one or plurality of electrodes comprise one or more of titanium, gold, stainless steel, platinum, iridium, tungsten, carbon fiber, silver, or silver chloride. In some embodiments, the one or plurality of electrodes are microelectrodes.

In some embodiments, the hydrogel matrix comprises a hydrogel of a first polymer that comprises a stiffness sufficient to prevent growth and/or cell migration and a hydrogel of a second polymer that comprises a stiffness sufficient to allow axon growth and/or cell migration. In some embodiments, the hydrogel matrix comprises a first polymer comprising no greater than about 15% PEG and from about 0.05% to about 5.0% of one or a combination of self-assembling peptides chosen from: RAD 16-I, RAD 16-II, EAK 16-I, EAK 16-II, and dEAK 16, and gelatin methacrylate.

In some embodiments, the permeable solid support comprises a plurality of pores from about 0.1 μm to about 3 μm in diameter. In some embodiments, the permeable solid support comprises polyester or polyvinyl polymers.

In some embodiments, the hydrogel matrix comprises one or a combination of compounds chosen from: polyethylene glycol (PEG), Puramatrix, methacrylated hyaluronic acid, agarose, methacrylated heparin, pyrrole (Py), oxidized polypyrrole (Ppy), and methacrylated dextran. In some embodiments, the hydrogel matrix comprises polyethylene glycol (PEG) at a concentration of no more than about 20% weight to volume (w/v) of the solution. In some embodiments, the hydrogel matrix comprises at least one cell-penetrable polymer at a concentration of from about 0.1% to about 3.0% in weight to volume (w/v) of the solution.

In some embodiments, the one or plurality of electrodes are in a substantially horizontal orientation on a top surface of the permeable solid support.

In some embodiments, the one or plurality of electrodes comprise at least one stimulating electrode, at least one recording electrode, and at least one ground electrode. In some embodiments, the at least one stimulating electrode and the at least one recording electrode are at a distance of about 1 μm to about 1 cm apart. In some embodiments, the stimulating electrode and the recording electrode are orientated substantially parallel to and spaced from each other. In some embodiments, the ground electrode comprises a first portion oriented substantially parallel with and spaced from the stimulating electrode, and the ground electrode comprises a second portion oriented substantially perpendicularly relative to the stimulating electrode.

In some embodiments, the insert comprises a first stimulating electrode and a first recording electrode oriented substantially parallel to each other and disposed on one side of the permeable solid support, a second stimulating electrode and a second recording electrode oriented substantially parallel to each other and disposed on an opposing side of the permeable solid support, and a ground electrode disposed between the first and second stimulating electrodes.

In some embodiments, contact pads of the first stimulating and recording electrodes are oriented away from contact pads of the second stimulating and recording electrodes. In some embodiments, the contact pads of the first stimulating and recording electrodes are oriented away from contact pads of the second stimulating and recording electrodes by about 180°. In some embodiments, a contact pad of the ground electrode is oriented away from the contact pads of the first and second stimulating and recording electrodes by about 90°.

In some embodiments, the one or plurality of contact pads are electrically connected to the one or plurality of electrodes.

In some embodiments, the insert comprises one or a plurality of cells. In some embodiments, the one or a plurality of cells comprise one or a combination of cells and/or tissues chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, an astrocyte, an oligodendrocyte, a Schwann cell, a microglial cell, an ependymal cell, a radial glial cell, a satellite cell, an enteric glial cell, a pituicyte, an immune cell, a dorsal root ganglia, and combinations thereof.

In some embodiments, the insert comprises a culture medium.

The present disclosure also relates to an adapter comprising: (i) a body defining a substantially flat and planar configuration with a top surface and a bottom surface; (ii) one or plurality of planar electrodes on the top surface of the body; (iii) a layer of insulating material; and (iv) a circular or substantially cylindrical collar positioned on its edge around a central opening formed and extending through the body.

In some embodiments, the body comprises a polymer resin. In some embodiments, the body comprises a first side edge and a second side edge, each dimensioned about 49 mm. In some embodiments, the body comprises a height dimensioned about 1 mm. In some embodiments, a central opening formed and extending through the body.

In some embodiments, the adapter comprises a pattern of contact pins radially disposed around the central opening and extending through the body, each of the contact pins electrically connected to at least one of the planar electrodes. In some embodiments, the one or plurality of planar electrodes are disposed on the top surface of the body in a substantially square pattern spaced from a perimeter of the body. In some embodiments, the pattern of the one or plurality of planar electrodes surrounds the central opening.

In some embodiments, the one or plurality of planar electrodes are configured to be attached to contacts of a plunger plate, the one or plurality of planar electrodes operably and electrically connected to an amplifier and current source through the contacts of the plunger plate.

In some embodiments, the one or plurality of planar electrodes form a continuous electrical connection perimeter along the top surface of the body.

The present disclosure also relates to a system comprising: (i) an insert positioned within the central opening; (ii) an adapter; (iii) an amplifier comprising a generator for electrical current; and (iv) a voltmeter and/or ammeter; wherein the amplifier, voltmeter and/or ammeter, and electrodes are electrically connected to each other via a circuit.

In some embodiments, the system comprises one or a combination of: controller, a recording device, a computer storage memory and a screen; wherein the screen if connected to the voltmeter and/or ammeter and is capable of displaying recording measurements from the one or plurality of electrodes.

The present disclosure also relates to a system comprising: (i) an insert; and (ii) a tissue culture support configured and dimensioned to receive the insert.

In some embodiments, the tissue culture support comprises a single well, and the insert is configured and dimensioned to be at least partially introduced into the single well. In some embodiments, the tissue culture support comprises a multiwell plate comprising 6, 12, 24 or 48 wells.

The present disclosure also relates to a method of producing a three-dimensional culture of one or a plurality of cells in a vessel. In some embodiments, the method comprises (i) contacting one or a plurality of cells with the permeable solid support of the insert; (ii) seeding one or a plurality of isolated cells or tissue explants comprising cells to the vessel of the insert; and (iii) applying a cell medium into the vessel with a volume of cell medium sufficient to cover the cells.

The present disclosure also relates to a method of testing of one or a plurality of cells, comprising: positioning the one or plurality of cells on the permeable solid support of an insert; applying an input current or voltage to the one or plurality of electrodes of the insert; and recording an output characteristic associated with the one or plurality of cells.

In some embodiments, the output characteristic comprises at least one of resistance or output current. In some embodiments, the method comprises comparing the input current or voltage to the output characteristic.

The present disclosure also relates to a method of testing of one or a plurality of cells, comprising: positioning the one or plurality of cells on the top surface of an adapter; applying an input current to the one or plurality of planar electrodes of the adapter; and recording an output characteristic associated with the one or plurality of cells.

The present disclosure also relates to a system comprising: a testing rig configured to receive an insert, the testing rig comprising a body with a housing and an inner passage extending through the housing; a plunger movably disposed within the inner passage and configured to be positioned in a raised position spaced from the insert or a lowered position disposed against the insert.

In some embodiments, the testing rig comprises a base with two aligners extending therefrom, the aligners configured to receive and maintain an orientation of the insert. In some embodiments, the base comprises a slot extending therethrough and the testing rig comprises a slide configured to be positioned within the slot of the base. In some embodiments, the testing rig comprises a spring disposed between the plunger and the housing, the spring urging the plunger towards the insert. In some embodiments, the plunger is configured to travel along a vertical axis between the raised and lowered positions. In some embodiments, the plunger comprises a bottom end with a plate and a rod extending perpendicularly from the bottom end. In some embodiments, the plate of the plunger comprises a circuit board with electrical contacts configured to be placed in electrical contact with the electrodes of the insert.

In some embodiments, the system further comprises at least one or combination of: a recording device, an amplifier, an electricity source, a controller, a user interface, a voltmeter, and an ammeter electrically connected to the testing rig.

The present disclosure also relates to a system comprising: (i) an insert; (ii) an adapter; and (iii) at least one of an amplifier comprising a generator for electrical current, a voltmeter or an ammeter; wherein the electrodes of the insert are electrically connected to the electrodes of the adapter; and wherein the electrodes of the adapter are operably linked to a circuit and at least one of the amplifier, the voltmeter, or the ammeter.

The present disclosure also relates to a method of assessing a response from one or more cells comprising: (a) growing one or more cells on the permeable solid support of an insert; (b) positioning the insert into an adapter; (c) placing the adapter in a system; (d) introducing one or more stimuli to the one or more cells; and (e) measuring one or more responses from the one or more cells to the one or more stimuli.

The present disclosure also relates to a method of evaluating the toxicity of an agent comprising: (a) culturing one or more cells and/or one or more tissue explants on the permeable solid support of an insert; (b) exposing at least one agent to the one or more cells and/or one or more tissue explants; (c) measuring and/or observing one or more morphometric changes of the one or more cells and/or one or more tissue explants; and (d) correlating one or more morphometric changes of the one or more cells and/or one or more tissue explants with the toxicity of the agent, such that, if the morphometric changes are indicative of decreased cell viability, the agent is characterized as toxic and, if the morphometric changes are indicative of unchanged or increased cell viability, the agent is characterized as non-toxic.

The present disclosure also relates to a method of measuring myelination or demyelination of one or more axons of one or a plurality of neuronal cells and/or one or a plurality of tissue explants, said method comprising:(a) culturing one or more neuronal cells and/or one or a plurality of tissue explants on the permeable solid support of an insert for a time and under conditions sufficient to grow at least one axon; and (b) detecting the amount of myelination on one or a plurality of axons of the one or more neuronal cells and/or one or more tissue explants.

The present disclosure also relates to a method of measuring myelination or demyelination of one or more axons of one or a plurality of neuronal cells and/or one or a plurality of tissue explants, said method comprising:(a) culturing one or more neuronal cells and/or one or a plurality of tissue explants on the permeable solid support of an insert for a time and under conditions sufficient to grow at least one axon; and (b) positioning the insert into an adapter; (c) inducing a compound action potential in the one or more neuronal cells and/or one or more tissue explants; (d) measuring the compound action potential; and (e) quantifying the levels of myelination of such one or more neuronal cells based on the compound action potential.

### BRIEF DESCRIPTION OF DRAWINGS

FIG. 1 is a diagrammatic top view of an exemplary mask that is used to deposit the metal electrodes in the configuration wanted. The mask is designed to snap into the insert.

FIG. 2 is a diagrammatic top view of an exemplary electrode configuration, with dotted lines indicating where a hydrogel construct could be placed.

FIG. 3 is a diagrammatic top view of an exemplary insert including electrodes and two hydrogel matrix layers.

FIG. 4A-FIG. 4C are a diagrammatic perspective view of an exemplary adapter configured to be implemented with an insert (FIG. 4A), a diagrammatic assembly of an insert and an adapter (FIG. 4B), and a diagrammatic, exploded view of an assembly of an insert and an adapter (FIG. 4C).

FIG. 5 is a diagrammatic perspective view of an exemplary system for electrophysiological examination of an insert.

FIG. 6A-FIG. 6C are pictures of a prototype of a permeable MEA device. FIG. 6A is a picture of an exemplary insert with deposited multielectrode pattern. FIG. 6B is a photograph of planar electrodes fabricated with permeable solid substrates on a support ring. Gold microelectrodes and reference electrodes are visible. Hydrogel micropatterns were fabricated directly on top of electrodes. FIG. 6C is a picture showing how permeable MEA devices are designed to fit inside conventional 6-well culture plates.

FIG. 7 is an image of an MEA insert with hydrogel constructs loaded into a custom electrophysiology rig.

FIG. 8 is a series of graphs showing resistivities of hydrogels in the low (left panel) and high (right panel) frequency domains. *** indicates p≤0.001.

FIG. 9 is a graph showing phase angles of hydrogels in the low-frequency domain. ** indicates p≤0.01, *** indicates p≤0.001.

FIG. 10 is a close-up picture of planar electrodes fabricated on permeable supports and containing dorsal root ganglion (DRG) tissue in a hydrogel matrix, used to obtain recordings of compound action potentials.

FIG. 11 is a chart of voltage versus time, depicting characteristic biological responses. Left: characteristic negative response from S2-3-1. Right: characteristic positive response from S4-5-2. The starting peak is the stimulus artifact.

FIG. 12 is a chart of voltage versus time, showing full pulse train electrophysiology data from constructs S2-3-1 (top series) and S2-3-2 (bottom series). Responses are indicated with stars. For the baseline response, only 32 stimuli were conducted due to visible fatigue. In the TTX response, ground voltage is seen floating in S2-3-1, however no response behavior is present. The clipped possible response in S2-3-1 in the post-TTX response was not counted.

### DETAILED DESCRIPTION OF EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

The term “plurality” as used herein is defined as any amount or number greater or more than 1.

As used herein, “substantially equal” means within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

As used herein, the terms “attach,” “attachment,” “adhere,” “adhered,” “adherent,” or like terms generally refer to immobilizing or fixing, for example, an electrode, a hydrogel, or a polymer, to a surface, such as by physical absorption, chemical bonding, and like processes, or combinations thereof.

The term “vessel” as used herein is any chamber, indentation, container, receptacle, or space. In some embodiments, a vessel is a well capable of holding no more than about 1,000, 900, 800, 700, 600, 500, 400, 300, 200, 100, 90, 80, 70, 60, 50, 40, 30, 20, 15, 12, 10, 9, 8, 7, 6, 5, 4, 3, 2, or 1 μL of total volume. In some embodiments, the vessel comprises the first and second cavities separated by a membrane and each of the first or second cavities is no more than about 100, 90, 80, 70, 60, 50, 40, 30, 20, 15, 12, 10, 9, 8, 7, 6, 5, 4, 3, 2, or 1 mL of total volume. In some embodiments, the total volume of the first and second vessels combined are no more than about 100, 90, 80, 70, 60, 50, 40, 30, 20, 15, 12, 10, 9, 8, 7, 6, 5, 4, 3, 2, or 1 mL of total volume. The insert, device or solid support disclosed herein can include multiple vessels in fluid communication with each other. In some embodiments, the insert, device or solid support comprises a detection vessel, which is configured to be near to substantially near one or a plurality of electrodes or some other disclosed device capable of stimulating the contents of the vessel and enabling detection of recordings in the vessel. In some embodiments, the insert, device or solid support comprises a reagent conduit, which may be branched or unbranched, linear, curved, or not linear, that connects the reaction vessel to the detection vessel. In some embodiments, at least a portion of the reagent conduit comprises at least one, two or more components of cell media, in solid form such as a powder or liquid form. The vessel or vessels may include a cavity defined by about 5 or about 10 or about 50 milliliters in volume. In some embodiments, the vessel is from about 1 milliliter to about 50 microliters in volume. In some embodiments, the vessel is from about 5 microliters to about 40 microliters in volume. In some embodiments, the vessel is from about 500 microliters to about 30 milliliters in volume. The vessel or vessels may include one or a plurality of hydrogel formations within the vessel cavity, and the hydrogel formation may comprise a further cavity into which biological samples, environmental samples or cells may be seeded. The hydrogel formation may be any size of dimension compatible with the vessel size. The hydrogel matrix, in some embodiments, may be a uniformly dimensioned layer that covers all or a portion of the bottom surface of the vessel. Three dimensional shapes such as cylinders, rectangular prism-like structures or elongated elliptical structures are contemplated by these embodiments.

The term “culture vessel” as used herein is defined as any vessel suitable for growing, culturing, cultivating, proliferating, propagating, or otherwise similarly manipulating cells. A culture vessel may also be referred to herein as a “culture insert” or “insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores, in some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated glial cells. In some embodiments, the culture vessel comprises a hydrogel and one or a plurality of isolated glial cells, to which one or a plurality of neuronal cells are seeded.

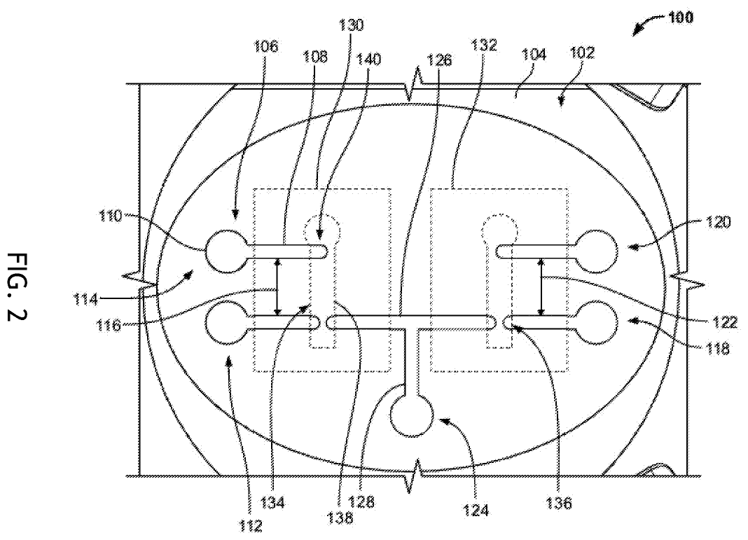

FIG. 2 is a diagrammatic top view of an exemplary insert 100 configured to be implemented with the system disclosed herein. The insert 100 generally includes a support 102 with a substantially flat or planar top surface 104 and an opposing bottom surface. The support 102 can be a permeable solid support (e.g., a permeable cell culture support or membrane) configured to receive one or more cells therein. In some embodiments, the support 102 can include a plurality of pores from about 0.2 μm to about 0.6 μm in diameter. In some embodiments, the support 102 can include a plurality of pores about 0.4 μm in diameter. In some embodiments, the support 102 can be fabricated from polyester. A pattern of a plurality of electrodes 106 can be physically attached (e.g., printed, or the like) to the top surface 104 of the insert 100. Although illustrated as having five electrodes 106, it should be understood that the insert 100 can include any number of electrodes 106 in a variety of patterns. Thus, any type and/or pattern of multielectrode arrays can be fabricated on the top surface 104 of the insert 100.

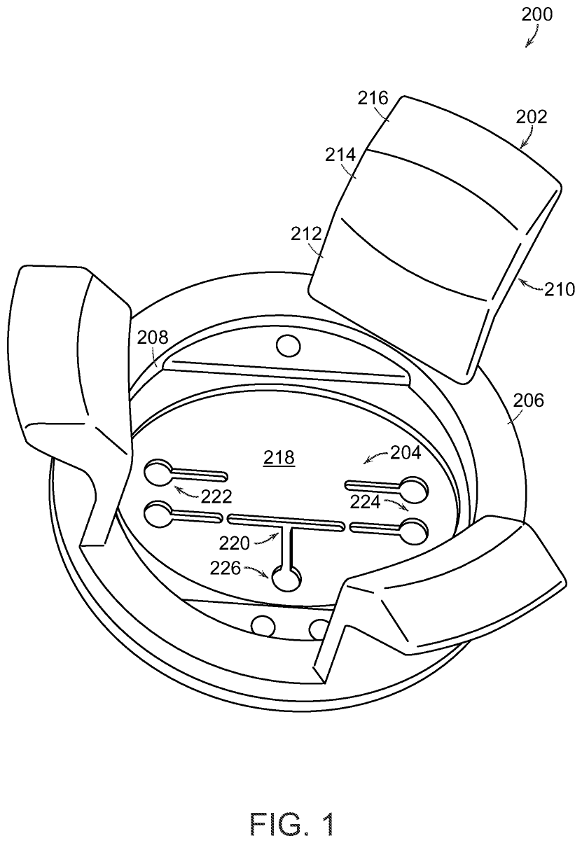

In some embodiments, e-beam vacuum evaporation, physical vapor deposition, and/or snap-in mask techniques can be used to physically attach the electrodes 106 to top surface 104 of the insert 100. For example, in some embodiments, a mask assembly 200 discussed above and illustrated in FIG. 1 can be used to physically attach the electrodes 106 to the insert 100 using e-beam vacuum evaporation. The mask assembly 200 can include a frame 202 and an interchangeable mask 204. The frame 202 can detachably receive and engage the mask 204 to ensure proper alignment of the mask 204 and insert 100.

The frame 202 includes a substantially cylindrical platform 206 forming a perimeter of the frame 202, and a central opening 208 extending therethrough. The frame 202 includes three stalks 210 radially spaced by approximately 120° relative to each other. The stalks 210 each include a portion 212 substantially perpendicular to the platform 206, an intermediate portion 214 angled relative to the portion 212, and a distal portion 216 angled further relative to the portion 214. The stalks 210 can be configured and dimensioned to snap into solution access holes of the insert 100.

The mask 204 includes a substantially planar body 218 with patterned holes 220 formed therein. The patterned holes 220 correspond with the desired electrode pattern to be physically attached to the insert 100. In the example shown in FIG. 1, the patterned holes 220 include two pairs of holes 222, 224 on opposing sides of each other, and a T-shaped hole 226 between the holes 222, 224. As will be discussed in greater detail below, the pairs of holes 222, 224 can correspond with stimulating and recording electrodes 106 of the insert 100, and the hole 226 can correspond with a ground electrode 106 of the insert 100.

The elongated shape and round endpoint of each of the holes 220 allows for formation of the electrode and a mating pad at the end of each electrode. The stalks 210 of the assembly 200 can be fabricated from plastic (e.g., acrylonitrile butadiene styrene (ABS), or the like) to allow for flexing of the material. The insert 100 can be snapped between the stalks 210 to engage the assembly 200 with the insert 100. E-beam vacuum evaporation can then be used to deposit metal (e.g., gold, titanium, stainless steel, platinum, iridium, tungsten, carbon fiber, silver, silver chloride, combinations thereof, or the like) onto the insert 100 to create the electrodes 106.

As an example, FIG. 2 shows a pattern of electrodes 106 corresponding to the mask 204 of FIG. 1. In some embodiments, the electrodes 106 can be microelectrodes. The electrodes 106 each include an elongated portion 108 and a round end 110 (e.g., a contact pad). The electrodes 106 are substantially flat and positioned adjacent to and/or against the top surface 104 such that a longitudinal axis of the electrodes 106 is parallel to the top surface 106. Thus, the electrodes 106 can be in a substantially horizontal orientation along the top surface 104 of the support 102. In some embodiments, the electrodes 106 can be protruding and/or three-dimensional, extending at varying angles or planes relative to the top surface 106. The electrodes 106 of FIG. 2 include a first stimulating electrode 112 and a first recording electrode 114 disposed substantially parallel to each other and spaced from each other by a distance 116 (e.g., about 1 μm to about 1 cm). Thus, the electrodes 112, 114 on their own are not electrically connected to each other. The ends 110 of the electrodes 112, 114 are oriented towards the perimeter of the insert 100.

The insert 100 of FIG. 2 includes a second stimulating electrode 118 and a second recording electrode 120 disposed substantially parallel to each other and spaced from each other by a distance 122. The distance 122 can be substantially equal to the distance 116, and the electrodes 118, 120 can be parallel to the electrodes 112, 114. The electrodes 118, 120 are therefore not electrically connected to each other, or the electrodes 112, 114. The ends 110 of the electrodes 118, 120 face away from the electrodes 112, 114 and are oriented towards the opposing side of the perimeter of the insert 100.

The insert 100 includes a ground electrode 124 disposed between the electrodes 112, 118. The electrode 124 can define a substantially T-shaped configuration, with first portion 126 extending parallel and in-line with the electrodes 112, 118 and a second, perpendicular portion 128 extending perpendicularly to the portion 126. The end 110 of the electrode 124 is located at the perpendicular portion 128 and oriented towards the perimeter of the insert 100. The electrode 124 is initially not electrically connected to the electrodes 112, 114, 118, 120.

One or more hydrogel matrix layers 130, 132 can be positioned on and at least partially affixed to the top surface 104 of the insert 100. In some embodiments, the hydrogel matrix layers 130, 132 can include one or a combination of compounds not limited to polyethylene glycol (PEG), Puramatrix, methacrylated hyaluronic acid, agarose, methacrylated heparin, pyrrole (Py), oxidized polypyrrole (Ppy), methacrylated dextran, or the like. The hydrogel matrix layer 130, 132 at least partially covers the electrodes 106. In some embodiments, rather than two separate or spaced hydrogel matrix layers 130, 132, a single hydrogel matrix layer can be used. For example, a support ring can be used to define the boundaries of the hydrogel matrix layer over a portion of the electrodes 106. The hydrogel matrix layer 130, 132 extends over and above the top surface 104 of the insert 100.

The hydrogel matrix layer 130, 132 includes at least one cavity 134, 136 (e.g., a keyhole shaped cavity) extending from the top of the hydrogel matrix layer 130, 132 to the top surface 104 and/or the electrode 106 of the insert 100. Each cavity 134, 136 includes a contiguous side region 138 and a bottom region 140. In some embodiments, the thickness of the hydrogel matrix layer 130, 132 can be from about 50 microns to about 500 microns. In some embodiments, the side region 138 can have a height from about 5 microns to about 50 microns. In some embodiments, the bottom region 140 can have a surface area of about 1 mm² to about 5 mm².

In some embodiments, the electrode 106 is positioned below the bottom region 140 of the cavity 134, 136. In some embodiments, the electrode 106 protrudes just above the bottom region 140. The cavity 134 provides a space electrically connecting the electrode 114 to the electrode 112, and the electrode 112 to the ground electrode 124. The cavity 136 provides a space electrically connecting the electrode 120 to the electrode 118, and the electrode 118 to the ground electrode 124. The combination of the cavity 134, 136 and the electrodes connected to the respective cavity can define a neurite construct of the insert 100.

As discussed herein, the insert 100 can be placed within a well of a multiwell culture plate and cell cultures can be placed and/or grown within the cavity 134, 136. The stimulating electrodes 112, 118 can be connected to an electrical source (e.g., via a controller, amplifier, user interface, voltmeter, combinations thereof, or the like). The recording electrodes 114, 120 and the ground electrode 124 can be connected to an electrophysiological examination system. Thus, current can be supplied to the cells within the cavities 134, 136 via the stimulating electrodes 112, 118, the cells provide an electrical connection between the electrodes 106 within the cavities 134, 136, and certain electrical characteristics (e.g., resistance, voltage drop, or the like) can be measured at the recording electrodes 114, 120 to determine the condition of the cells.

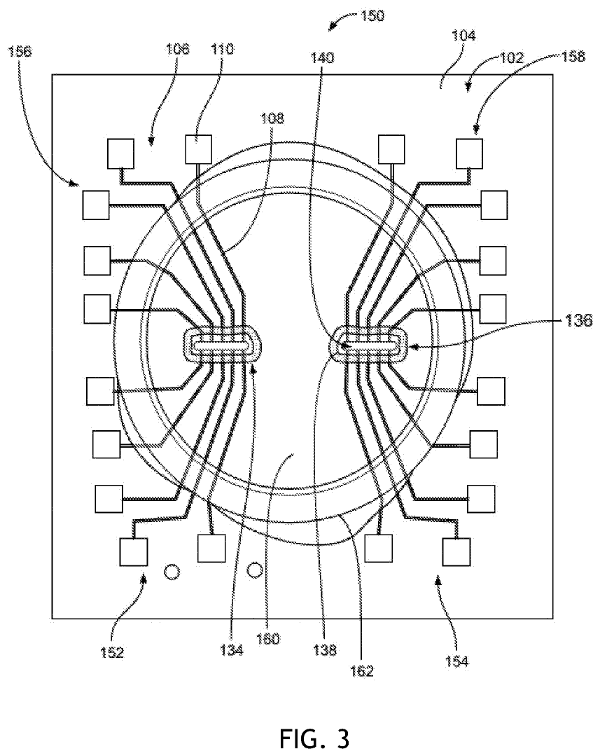

FIG. 3 is a top view of an exemplary insert 150. The insert 150 can be substantially similar in structure and/or function to the insert 100. Therefore, like reference numbers represent like structures. Rather than only including two pairs of electrodes 106, the insert 150 includes a pattern of multiple electrodes 106 on either side of the support 102. Although not shown, it should be understood that the insert 150 includes a ground electrode electrically connected to the electrodes 106. The electrodes 106 can include square ends 110 defining the contact pad for each of the electrodes 106. The top surface 104 defines a flat-bottomed portion onto which the electrodes 106 are positioned.

One set of electrodes 152, 154 on each side of the insert 150 can be used as the stimulating electrode, while the opposing set of electrodes 156, 158 can be used as the recording electrodes. The insert 150 includes a single hydrogel matrix layer 160 affixed to the top surface 104 of the insert 150. In some embodiments, the insert 150 can include a culture or support ring 162 that provides structural support and maintains the perimeter of the hydrogel matrix layer 160. The ring 162 can be physically attached to the top surface 104 by its edge, and extends by a height of approximately 15 millimeters from the top surface 104. Although shown as a substantially circular structure, it should be understood that the support ring 162 can be of any configuration. In some embodiments, the ring 162 can be dimensioned such that the insert 150 can be at least partially positioned into a well of a support plate. In some embodiments, the support plate can have a single well of approximately 3.5 cm in diameter, or can be a multiwell plate having 6 wells of approximately 3.46 cm in diameter, 12 wells of approximately 2.21 cm in diameter, or 24 wells of approximately 1.55 cm in diameter or 48 wells of approximately from about 0.1 to 1 cm in diameter. Thus, rings 162 of different dimensions can be used based on the type of well plate to be implemented with the insert 150.

The hydrogel matrix layer 160 includes two separate cavities 134, 136 extending through the hydrogel matrix layer 160 to the electrodes 106 and/or the top surface 104. The cells disposed within the cavities 134, 136 provide the inductive medium for electrical connection between the respective stimulating and recording electrodes on either side of the cavities 134, 136. Although shown with the hydrogel matrix layer 160, in some embodiment, the insert 150 can be implemented without the hydrogel matrix layer 160. For example, the insert 150 can be used to culture organotypic brain slices without the use of the hydrogel matrix layer 160.

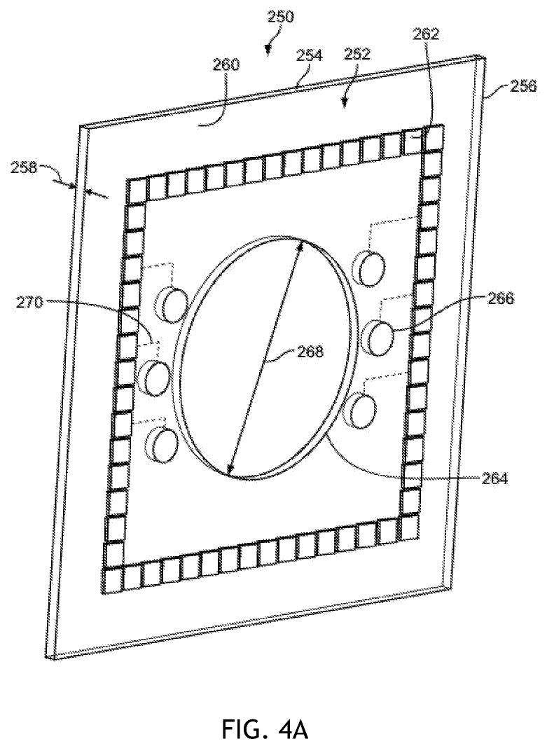

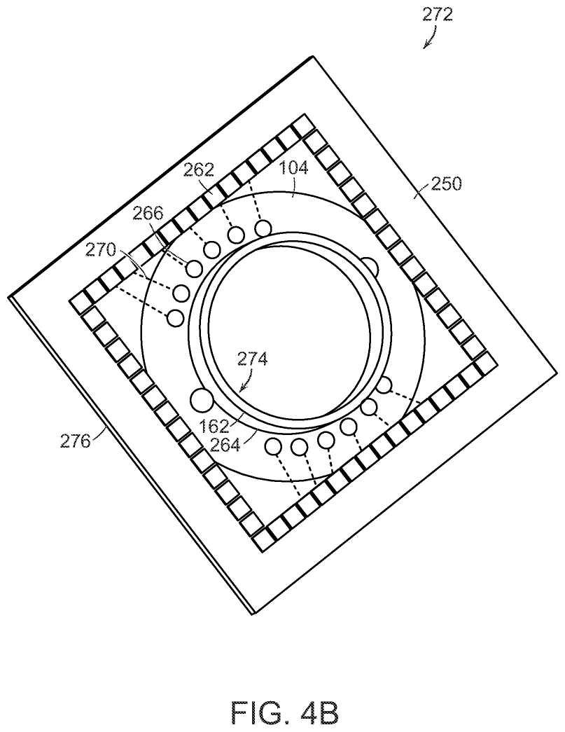

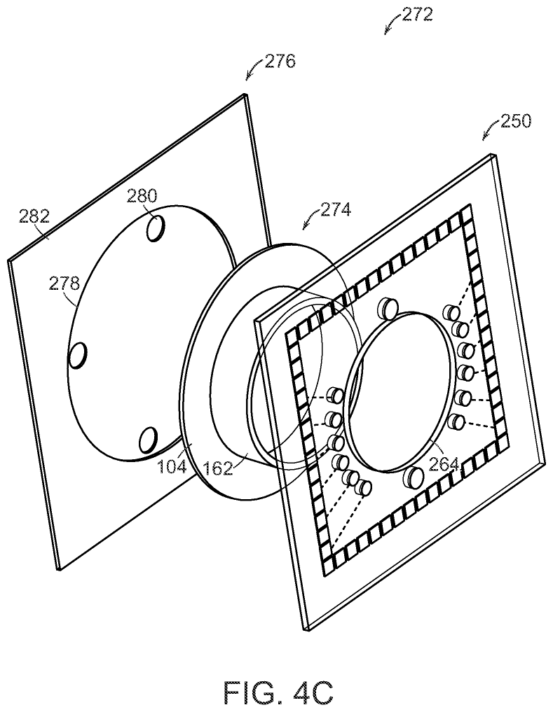

FIG. 4A is a diagrammatic perspective view of an exemplary adapter 250 (e.g., a collar) configured to be implemented with the inserts discussed herein. The adapter 250 generally includes a body 252 fabricated from a polymer resin. In some embodiments, the body 252 can be fabricated from two or more layers of materials coupled together. In some embodiments, the top surface 260 of the body 252 can be fabricated from a layer of insulating material to provide insulation between certain components or sections of the adapter 250 and the insert when the adapter 250 and insert are positioned against each other. The body 252 can be in a substantially square configuration. In some embodiments, the body 252 can be of any shape, e.g., square, rectangular, oval, circular, polygonal, or the like. In some embodiments, the side edges 254, 256 of the body 252 can be dimensioned as approximately 49 mm, and the height or thickness 258 of the body 258 can be dimensioned as approximately 1 mm. The body 252 defines a substantially planar or flat configuration having a top surface 260.

One or more electrodes 262 can be physically attached to the top surface 260 in a predetermined pattern. Each of the electrodes 262 can be substantially flat in configuration, and extends substantially parallel to the top surface 260. In some embodiments, the electrodes 262 can be protruding and/or three-dimensional, extending at varying angles or planes relative to the top surface 260. In some embodiments, each electrode 262 can define a substantially square configuration. In some embodiments, the pattern in which the electrodes 262 are disposed on the top surface 260 can define a square spaced from the perimeter edges 254, 256 of the body 252. In some embodiments, the pattern in which the electrodes 262 are disposed on the top surface 260 can be square, rectangular, oval, circular, polygonal, or the like. Particularly, the pattern of the electrodes 262 can be selected to correspond with contacts of testing equipment to create an electrical contact between the testing equipment and the electrodes 106 of the insert.

The adapter 250 includes a central opening 264 configured to receive therethrough the support ring 162 of the insert. The diameter 268 of the central opening 264 is therefore dimensioned to correspond with and receive therethrough the diameter of the support ring 162 of the insert. The adapter 250 includes one or more contact pins 266 disposed around the central opening 264 in a radial pattern. The contact pins 266 are disposed between the central opening 264 and the electrodes 262. Connecting pathways 270 electrically couple and/or connect the contact pins 266 and the electrodes 262. The contact pins 266 traverse the thickness or height 258 of the adapter 250, extending to the bottom surface of the adapter 250, and are configured to contact or mate against the ends 110 of the electrodes 106 on the insert. When the adapter 250 receives the support ring 162 through the central opening 264 and the bottom surface of the adapter 250 is positioned against the top surface of the insert, the contact pins 266 contact and create an electrical connection with the ends 110 of the electrodes 106 of the insert, and the pathways 270 electrically couple the contact pins 266 and the electrodes 262.

An electrical connection between the electrodes 106, 262 can thereby be achieved when the adapter 250 is positioned over the insert. It should be understood that any insulating layer of the adapter 250 only provides for insulation or protection to the remaining surfaces of the insert, while the electrodes 106, 262 remain exposed to achieve electrical contact. The adapter 250 can be electrically connected to electrophysiological examination equipment and acts as an intermediate connector such that current can be supplied to the insert from the electrophysiological examination equipment and measured to determine characteristics of the cells on the insert.

FIGS. 4B and 4C show a diagrammatic assembled view and a diagrammatic exploded view of an exemplary assembly 272 of an insert 274 and the adapter 250. The insert 274 includes the support ring 162 in which electrodes (and in some embodiments, a hydrogel) can be disposed. The body 104 of the insert 274 can define a substantially circular extension beyond the perimeter of the support ring 162. As noted above, the ends 110 (e.g., contact pads) of the electrodes of the insert 274 extend beyond the perimeter of the support ring 162 and are disposed along the top surface of the body 104 outside of the support ring 162. As shown in FIG. 4B, the support ring 162 passes through the central opening 264 of the adapter 250 such that the bottom surface of the adapter 250 mates against the top surface of the insert 274. Specifically, the contact pins 266 mate against and create an electrical connection with the ends 110 of the electrodes 106 disposed on the outside of the perimeter of the support ring 162. The contact pins 266 are electrically coupled to the electrodes 262 via pathways 270.

In some embodiments, a bottom plate 276 can be coupled to the bottom surface of the insert 274 and/or the adapter 250. The bottom plate 276 includes a body 282 having a substantially planar, square configuration. In some embodiments, the body 282 of the bottom plate 276 can be configured and dimensioned to correspond with the shape of the adapter 250. The bottom plate 276 can include a recessed section 278 configured substantially complementary to the bottom area of the insert 274 such that the position of the insert 274 relative to the bottom plate 276 can be maintained. In some embodiments, fasteners (not shown) can be passed through openings 280 of the bottom plate 276 to secure the bottom plate 276 to the insert 274.

The assembly 272 can be used with testing equipment to provide current to the cells in the insert 274. Particularly, current can be supplied from the testing equipment to the electrodes 262 of the adapter 250, passes from the electrodes 262 to the contact pins 266 through the pathways 270, passes from the contact pins 266 to the electrodes 106, and passes further into the cells. The output current can be received in reverse format from the electrodes 106, to the contact pins 266, to the electrodes 262, and output to the testing equipment to determine measured characteristics associated with the cells.

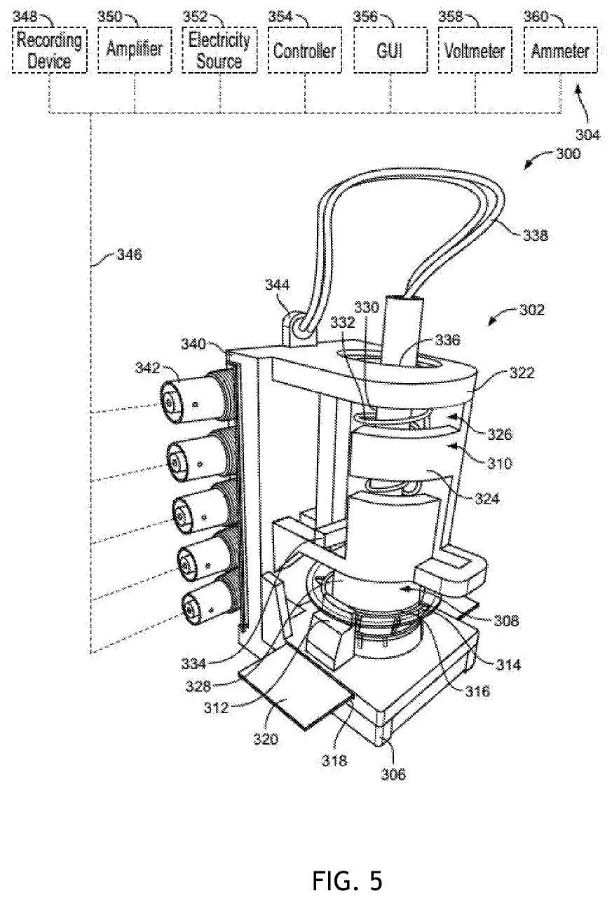

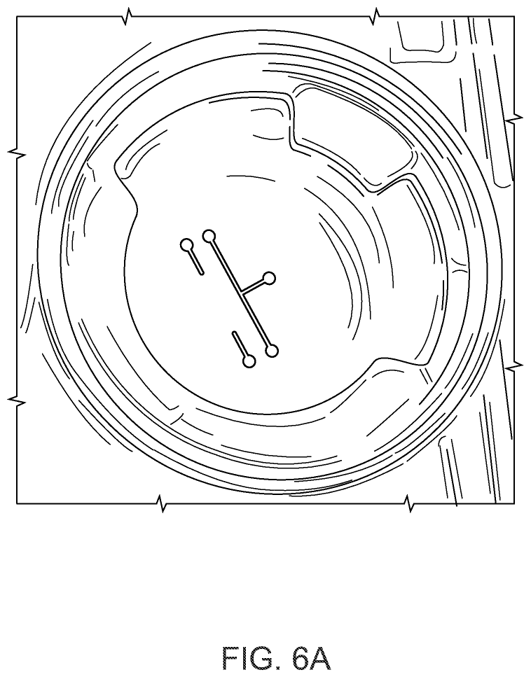

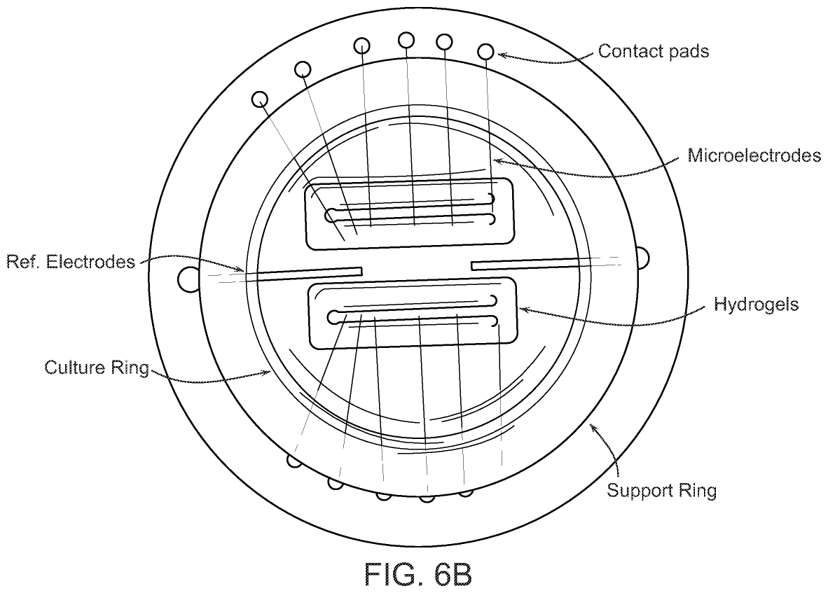

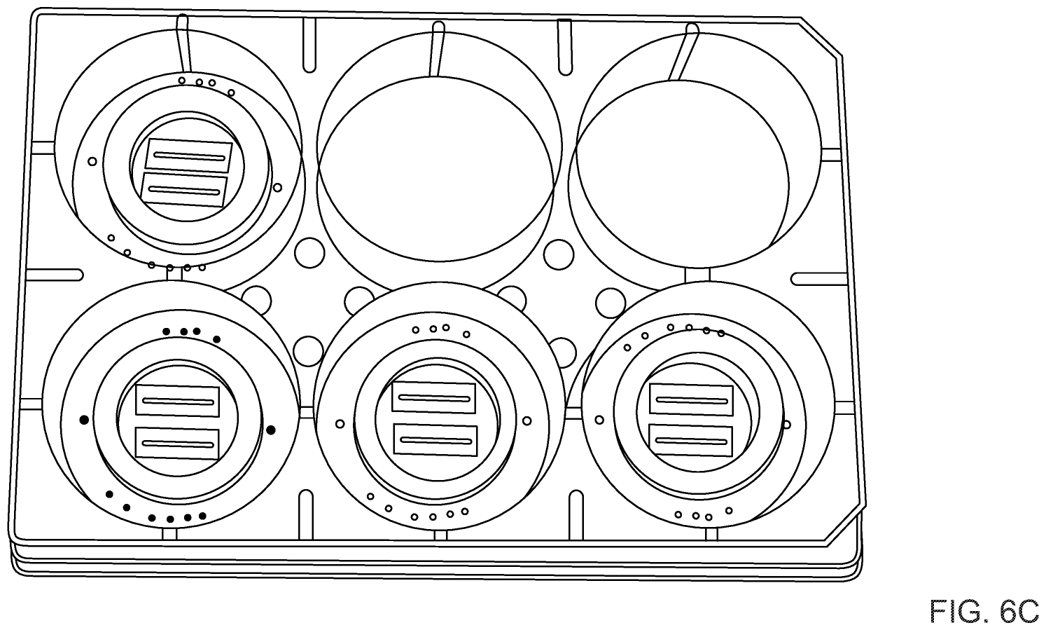

FIG. 5 is a diagrammatic perspective view of an exemplary system 300 for electrophysiological examination of the insert discussed herein. The system 300 includes a testing rig 302 and a plurality of components that collectively define an electrophysiology unit 304. The testing rig 302 is configured such that the inserts having patterned electrodes can efficiently have a continuous electrical connection formed between the ends of the electrodes directly in contact with neurite constructs, as well as the stimulating and recording electrophysiology unit 304.

The rig 302 includes a base 306, and a plunger 308 movably disposed within the main assembly or body 310 of the rig 302. The base 306 includes two insert flanges or aligners 312 on opposing sides of the rig 302. The aligners 312 ensure that the insert 314 placed in the rig 302 is maintained in the correct or desired orientation relative to fluid access holes. The bottom end of the plunger 308 includes a plate 316 with electrical contacts corresponding to the electrodes contact pads (e.g., ends 110) of the insert 314. The aligners 312 therefore ensure that the electrical contacts of the plate 316 mate with the corresponding electrode mating pads in the insert 314 when the plate 316 is brought and positioned against the insert 314. The base 306 includes a slot 318 configured to receive therethrough a glass slide 320. The slide 320 provides a flat, cleanable surface on which the insert 314 can rest during testing. The base 306 can be detachable from the body 310 to provide clearance for the plunger 308 to be inserted.

The body 310 provides stability to the rig 302 and holds the spring-loaded plunger 308 above the insert 314. The body 310 includes a substantially cylindrical housing 322 with an inner passage 326 in which the plunger 308 travels along a vertical axis, and a plurality of perpendicular slots 324 for constraining the vertical travel of the plunger 308. The plunger 308 includes a bottom end 328 defining a substantially cylindrical configuration and a rod 330 extending perpendicularly from the bottom end 328. The diameter of the rod 330 is dimensioned smaller than the diameter of the bottom end 328.

A conical compression spring 332 is disposed around the rod 330, with one end positioned against the inner top surface of the housing 322 and the opposing end positioned against the top surface of the bottom end 328. The spring 332 thereby provides a force against the bottom end 328, urging the bottom end 328 (e.g., the plate 316) downward against the insert 314. The rig 302 can include a locking mechanism (e.g., pin 334) for locking the plunger 308 in a raised position (e.g., raised above the insert 314). Removing the base 306 allows for the plunger 308 and spring 332 to be inserted into the passage 326 during assembly of the rig 302.

The plate 316 can include a circuit board with gold-plated contacts configured to be placed in electrical contact (directly or indirectly) with the electrodes of the insert 314. In some embodiments, an adapter (e.g., adapter 250) can be disposed under the plate 316 of the plunger 308 (with or without the insert 314) to provide an interface for creating an electrical contact between the contacts of the plunger 308 and the electrodes of adapter (or the insert 314). If used with the insert 314, the adapter provides a means of ensuring an electrical contact between the plunger 308 and insert 314 even if the insert 314 has varying patterns of electrodes by first creating an electrical contact between the electrodes of the insert 314 and the adapter. The adapter can include a pattern of electrodes that is compatible with the contacts of the plate 316. Thus, the adapter acts as an interface to ensure compatibility between the plate 316 and the insert 314.

The spring 322 provides a downward force on the plunger 308 to ensure a continuous pressure connection between the contacts of the plate 316 and the electrodes of the insert 314. The top of the rod 330 extends through an opening 336 and above the top surface of the housing 322. The rod 330 is hollow, allowing for wiring 338 to pass from the circuit board on the plate 316, through the plunger 308, and electrically connect to the electrophysiology unit 304. The wiring 338 electrically connects to the contacts of the plate 316 of the plunger 308 such that stimulating current can be supplied to the insert 314. The hollow rod 330 ensures that the plunger 308 can move up and down consistently without interference from the wiring 338.

In some embodiments, a single stimulus connection can be attached to the contacts of the plate 316 such that identical stimuli are continuously delivered to each of the two constructs or cavities of the insert 314. The rig 302 includes a board 340 secured to the body 310 and configured to support a plurality of jacks 342 (e.g., Bayonet Neill-Concelman (BNC) jacks). The wiring 338 extending from the plunger 308 electrically connects with the jacks 342 via an interface 344. One or more of the jacks 342 can be electrically connected to the electrophysiology unit 304 using wiring 346.

The electrophysiology unit 304 can include a recording device 348, an amplifier 350, an electricity source 352, a controller 354, a graphical user interface (GUI) 356, a voltmeter 358, an ammeter 360, combinations thereof, or the like. In some embodiments, the amplifier 350 can include a generator acting as the source of electrical current for the electrophysiology unit 304. Each of the components of the electrophysiology unit 304 can be electrically connected to each other via the wiring 346 and/or one or more circuits.

The term “electrical stimulation” refers to a process in which the cells are being exposed to an electrical current of either alternating current (AC) or direct current (DC). The current may be introduced into the solid substrate or applied via the cell culture media or other suitable components of the cell culture system. In some embodiments, the electrical stimulation is provided to the device or system by one or a plurality of electrodes at different positions within the device or system to create a voltage potential across the cell culture vessel. The electrodes are in operable connection with one or a plurality of amplifiers, voltmeters, ammeters, and/or electrochemical systems (such as batteries or electrical generators) by one or a plurality of wires. Such devices and wires create a circuit through which an electrical current is produced and by which an electrical potential is produced across the cell culture system.

Most planar microelectrode arrays (MEAs) are designed to be multiple-use devices. In conventional applications, cells are cultured on top of the planar MEAs, and when the experiment is finished, the cells can be removed, devices washed, residual organic matter removed with plasma treatment, and then the devices reused several times. With 3D and permeable-substrate MEAs, where the hydrogels and tissues are integrated snugly into the devices, it may not be possible to reuse the MEAs. Thus, the low-cost fabrication processes proposed within are critical innovations making this approach feasible on a commercial scale. In some embodiments, disposable, single-use devices can be shipped directly to customers as kits for incorporation of tissue into the devices. Such a device would be the first of its kind offering 3D tissue architecture mimicking the anatomy of the nervous system, all integrated “on-a-chip”.

In some embodiments, a thin (˜10 μm), transparent polyester sheet with 0.4 μm pores (SABEU GmbH & Co., Germany) will be used to fabricate an insert. This is the same material used by Corning to manufacture their Transwell® permeable culture supports. A stainless-steel shadow mask, fabricated with electron-beam lithography, will be used to direct metallization of the electrode pattern using electron-beam vapor deposition. A layer of titanium will facilitate adhesion to the polymer membrane followed by a layer of gold. This process has been optimized to reduce heat, which is essential to maintain the integrity of the porous membrane. Sheets with electrode patterns will be secured with adhesive to a plastic support ring to prevent wrinkling while stamping out and affixing a glass or polystyrene culture ring with adhesive. After fabrication processes are complete, devices will be sterilized with oxygen plasma treatment. Hydrogel micropatterns will be fabricated directly on top of the permeable supports with deposited electrodes. This process is effective for producing inserts of planar electrodes on permeable substrates, the inserts designed to fit directly within culture plates for culturing tissue, and then to be removed for electrophysiological recording. Fabrication of an adapter will enable use with commercial MEA equipment.

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. Hydrogels and hydrogel matrices are known in the art and various types have been described, for example, in U.S. Pat. Nos. 5,700,289, and 6,129,761; and in Curley and Moore, 2011; Curley et al., 2011; Irons et al., 2008; and Tibbitt and Anseth, 2009; each of which are incorporated by reference in their entireties. In some embodiments, the hydrogel or hydrogel matrix can be solidified by subjecting the liquefied pregel solution to ultraviolet light, visible light or any light above about 300 nm, 400 nm, 450 nm or 500 nm in wavelength. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, cells are incorporated in the hydrogel or hydrogel matrices. In some embodiments, cells from nervous system are incorporated into the hydrogel or hydrogel matrices. In some embodiments, the cells from nervous system are Schwann cells and/or oligodendrocytes. In some embodiments, the hydrogel or hydrogel matrix comprises tissue explants from the nervous system of an animal, (such as a mammal) and a supplemental population of cells derived from the nervous system but isolated and cultured to enrich its population in the culture. In some embodiments, the hydrogel or hydrogel matrix comprises a tissue explant such as a retinal tissue explant, DRG, or spinal cord tissue explant and a population of isolated and cultured Schwann cells, oligodendrocytes, and/or microglial cells. In some embodiments, two or more hydrogels or hydrogel matrixes are used simultaneously in the cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed cell culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “cell-penetrable polymer” refers to a hydrophilic polymer, with identical or mixed monomer subunits, at a concentration and/or density sufficient to create spaces upon crosslinking in a solid or semisolid state on a solid substrate, such space are sufficiently biocompatible such that a cell or part of a cell can grow in culture.

The term “cell-impenetrable polymer” refers to a hydrophilic polymer, with identical or mixed monomer subunits, at a concentration and/or density sufficient to, upon crosslinking in a solid or semisolid state on a solid substrate, not create biocompatible spaces or compartments. In other words, an cell-impenetrable polymer is a polymer that, after crosslinking at a particular concentration and/or density, cannot support growth of a cell or part of a cell in culture.

One of ordinary skill can appreciate that a cell-impenetrable polymer and a cell-penetrable polymer may comprise the same or substantially the same polymers but the difference in concentration or density after crosslinking creates a hydrogel matrix with some portions conducive to grow a cell or part of cell in culture.

In some embodiments, the hydrogel or hydrogel matrixes can have various thicknesses. In some embodiments, the thickness of the hydrogel or hydrogel matrix is from about 100 μm to about 800 μm. In some embodiments, the thickness of the hydrogel or hydrogel matrix is from about 150 μm to about 800 μm. In some embodiments, the thickness of the hydrogel or hydrogel matrix is from about 200 μm to about 800 μm. In some embodiments, the thickness of the hydrogel or hydrogel matrix is from about 250 μm to about 800 μm. In some embodiments, the thickness of the hydrogel or hydrogel matrix is from about 300 μm to about 800 μm. In some embodiments, the thickness of the hydrogel or hydrogel matrix is from about 350 μm to about 800 μm. In some embodiments, the thickness of the hydrogel or hydrogel matrix is from about 400 μm to about 800 μm. In some embodiments, the thickness of the hydrogel or hydrogel matrix is from about 450 μm to about 800 μm. In some embodiments, the thickness of the hydrogel or hydrogel matrix is from about 500 μm to about 800 μm. In some embodiments, the thickness of the hydrogel or hydrogel matrix is from about 550 μm to about 800 μm. In some embodiments, the thickness of the hydrogel or hydrogel matrix is from about 600 μm to about 800 μm. Iⁿ some embodiments, the thickness of the hydrogel or hydrogel matrix is from about 650 μm to about 800 μm. In some embodiments, the thickness of the hydrogel or hydrogel matrix is from about 700 μm to about 800 μm. In some embodiments, the thickness of the hydrogel or hydrogel matrix is from about 750 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. Iⁿ some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. Iⁿ some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrixes can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

In some embodiments, the hydrogel or hydrogel matrix comprises one or more synthetic and/or natural polypeptides. In some embodiments, the hydrogel or hydrogel matrix comprises one or more of the following polypeptides: polylysine, polyglutamate or polyglycine. In some embodiments, the hydrogel comprises one or a combination of polymers select from those published in Khoshakhlagh et al., “Photoreactive interpenetrating network of hyaluronic acid and Puramatrix as a selectively tunable scaffold for neurite growth” Acta Biomaterialia, Jan. 21, 2015.

Any hydrogel suitable for cell growth can be formed by placing any one or combination of polymers disclosed herein at a concentration and under conditions and for a sufficient time period sufficient to create two distinct densities of crosslinked polymers: one cell-penetrable and one cell-impenetrable. The polymers may be synthetic polymers, polysaccharides, natural proteins or glycoproteins and/or polypeptides such as those selected from below.

### Synthetic Polymers

Such as polyethylene glycol (polyethylene oxide), polyvinyl alcohol, poly-2-hydroxyethyl methacrylate, polyacrylamide, silicones, their combinations, and their derivatives.

Polysaccharides (Whether Synthetic or Derived from Natural Sources)

Such as hyaluronic acid, heparan sulfate, heparin, dextran, agarose, chitosan, alginate, their combinations, and their derivatives.

### Natural Proteins or Glycoproteins

Such as collagen, gelatin, elastin, titin, laminin, fibronectin, fibrin, keratin, silk fibroin, their combinations, and their derivatives.

### Polypeptides (Whether Synthetic or Natural Sources)

Such as polylysine, and RAD and EAK peptides

In some embodiments, the disclosed insert and/or systems comprise a surface covered in a hydrogel matrix, the hydrogel matrix comprising a cell penetrable and cell impenetrable polymer, peptide network, sugar, cellulose matrix or combinations thereof. In some embodiments, the cell impenetrable hydrogel serves as a mold or base for the cell penetrable layer. In some embodiments, the cell impenetrable hydrogel comprises PEG diacrylate up to about 20% w/v (we use 8 or 10%) or gelatin methacrylate, dextran methacrylate, methacrylated hyaluronic acid, agarose, acrylamide, or the like. In some embodiments, the cell penetrable hydrogel comprises any of the cell impenetrable hydrogels mentioned above from about 0.1 to about 5% in weight to volume (w/v). In some embodiments, the cell penetrable hydrogel comprises about 4% gelatin methacrylate w/v and/or about 1% Matrigel at about 1%.

The term “isolated neurons” refers to neuronal cells that have been removed or disassociated from an organism or culture from which they originally grow. In some embodiments isolated neurons are neurons in suspension. In some embodiments, isolated neurons are a component of a larger mixture of cells including a tissue sample or a suspension with non-neuronal cells. In some embodiments, neuronal cells have become isolated when they are removed from the animal from which they are derived, such as in the case of a tissue explant. In some embodiments isolated neurons are those neurons in a DRG excised from an animal. In some embodiments, the isolated neurons comprise at least one or a plurality cells that are from one species or a combination of the species chosen from: sheep cells, goat cells, horse cells, cow cells, human cells, monkey cells, mouse cells, rat cells, rabbit cells, canine cells, feline cells, porcine cells, or other non-human mammals. In some embodiments, the isolated neurons are human cells. In some embodiments, the isolated neurons are stem cells that are pre-conditioned to have a differentiated phenotype similar to or substantially similar to a human neuronal cell. In some embodiments, the isolated neurons are human cells. In some embodiments, the isolated neurons are stem cells that are pre-conditioned to have a differentiated phenotype similar to or substantially similar to a non-human neuronal cell. In some embodiments, the stem cells are selected from: mesenchymal stem cells, induce pluripotent stem cells, embryonic stem cells, hematopoietic stem cells, epidermal stem cells, stem cells isolated from the umbilical cord of a mammal, or endodermal stem cells.

The term “neurodegenerative disease” is used throughout the specification to describe a disease which is caused by damage to the central nervous system ad or peripheral nervous system. Exemplary neurodegenerative diseases which may be examples of diseases that could be studied using the disclosed model, system or device include for example, Parkinson's disease, Huntington's disease, amyotrophic lateral sclerosis (Lou Gehrig's disease), Alzheimer's disease, lysosomal storage disease (“white matter disease” or glial/demyelination disease, as described, for example by Folkerth, J. Neuropath. Exp. Neuro., 58, 9, Sep. 1999), Tay Sachs disease (beta hexosamimidase deficiency), other genetic diseases, multiple sclerosis, brain injury or trauma caused by ischemia, accidents, environmental insult, etc., spinal cord damage, ataxia and alcoholism. In addition, the present invention may be used to test the efficacy, toxicity, or neurodegenerative effect of agents on neuronal cells in culture for the study of treatments for neurodegenerative diseases. The term neurodegenerative diseases also includes neurodevelopmental disorders including for example, autism and related neurological diseases such as schizophrenia, among numerous others.

The term “neuronal cells” as used herein are defined as cells that comprise at least one or a combination of dendrites, axons, and somata, or, alternatively, any cell or group of cells isolated from nervous system tissue. In some embodiments, neuronal cells are any cell that comprises or is capable of forming an axon. In some embodiments, the neuronal cell is a Schwann cells, glial cell, neuroglia, cortical neuron, embryonic cell isolated from or derived from neuronal tissue or that has differentiated into a cell with a neuronal phenotype or a phenotype which is substantially similar to a phenotype of a neuronal cell, induced pluripotent stem cells (iPS) that have differentiated into a neuronal phenotype, or mesenchymal stem cells that are derived from neuronal tissue or differentiated into a neuronal phenotype. In some embodiments, neuronal cells are neurons from dorsal root gangila (DRG) tissue, retinal tissue, spinal cord tissue, or brain tissue from an adult, adolescent, child or fetal subject. In some embodiments, neuronal cells are any one or plurality of cells isolated from the neuronal tissue of a subject. In some embodiments, the neuronal cells are mammalian cells. In some embodiments, the cells are human cells. In some embodiments, the cells are non-human mammalian cells or derived from cells that are isolated from non-human mammals. If isolated or disassociated from the original animal from which the cells are derived, the neuronal cells may comprises isolated neurons from more than one species.

In some embodiments, neuronal cells are one or more of the following: central nervous system neurons, peripheral nervous system neurons, sympathetic neurons, parasympathetic neurons, enteric nervous system neurons, spinal motor neurons, motor neurons, sensory neurons, autonomic neurons, somatic neurons, dorsal root ganglia, cholinergic neurons, GABAergic neurons, glutamatergic neurons, dopaminergic neurons, serotonergic neurons, interneurons, adrenergic neurons, and trigeminal ganglia. In some embodiments, glial cells are one or more of the following: astrocytes, oligodendrocytes, Schwann cells, microglia, ependymal cells, radial glia, satellite cells, enteric glial cells, and pituyicytes. In some embodiments, immune cells are one or more of the following: macrophages, T cells, B cells, leukocytes, lymphocytes, monocytes, mast cells, neutrophils, natural killer cells, and basophils. In some embodiments, stem cells are one or more of the following: hematopoietic stem cells, neural stem cells, embryonic stem cells, adipose derived stem cells, bone marrow derived stem cells, induced pluripotent stem cells, astrocyte derived induced pluripotent stem cells, fibroblast derived induced pluripotent stem cells, renal epithelial derived induced pluripotent stem cells, keratinocyte derived induced pluripotent stem cells, peripheral blood derived induced pluripotent stem cells, hepatocyte derived induced pluripotent stem cells, mesenchymal derived induced pluripotent stem cells, neural stem cell derived induced pluripotent stem cells, adipose stem cell derived induced pluripotent stem cells, preadipocyte derived induced pluripotent stem cells, chondrocyte derived induced pluripotent stem cells, and skeletal muscle derived induced pluripotent stem cells. In some embodiments, cells include other cell types such as keratinocytes, muscle cells, cardiac cells, or endothelial cells.

The terms “cell culture medium” or simply “culture medium” as used herein are defined as any nutritive substance suitable for supporting the growth, culture, cultivating, proliferating, propagating, or otherwise manipulating neuronal or other types of cells. In some embodiments, the medium comprises neurobasal medium supplemented with nerve growth factor (NGF). In some embodiments, the medium comprises fetal bovine serum (FBS). In some embodiments, the medium comprises L-glutamine. In some embodiments, the medium comprises ascorbic acid in a concentration ranging from about 0.001% weight by volume to about 0.01% weight by volume. In some embodiments, the medium comprises ascorbic acid in a concentration ranging from about 0.001% weight by volume to about 0.008% weight by volume. In some embodiments, the medium comprises ascorbic acid in a concentration ranging from about 0.001% weight by volume to about 0.006% weight by volume. In some embodiments, the medium comprises ascorbic acid in a concentration ranging from about 0.001% weight by volume to about 0.004% weight by volume. In some embodiments, the medium comprises ascorbic acid in a concentration ranging from about 0.002% weight by volume to about 0.01% weight by volume. In some embodiments, the medium comprises ascorbic acid in a concentration ranging from about 0.003%) weight by volume to about 0.01% weight by volume. In some embodiments, the medium comprises ascorbic acid in a concentration ranging from about 0.004% weight by volume to about 0.01% weight by volume. In some embodiments, the medium comprises ascorbic acid in a concentration ranging from about 0.006% weight by volume to about 0.01% weight by volume. In some embodiments, the medium comprises ascorbic acid in a concentration ranging from about 0.008% weight by volume to about 0.01% weight by volume. In some embodiments, the medium comprises ascorbic acid in a concentration ranging from about 0.002%) weight by volume to about 0.006% weight by volume. In some embodiments, the medium comprises ascorbic acid in a concentration ranging from about 0.003% weight by volume to about 0.005% weight by volume.

Cell culture media suitable for the methods of the present invention are known in the art and include, but are not limited to, BEGMTM Bronchial Epithelial Cell Growth medium, Dulbecco's Modified Eagle's Medium (DMEM), Dulbecco's Modified Eagle's Medium high glucose (DMEM-H), McCoy's 5A Modified Medium, RPMI, Ham's media, Medium 199, mTeSR, and so on. The cell culture medium may be supplemented with additional components such as, but not limited to, vitamins, minerals, salts, growth factors, carbohydrates, proteins, serums, amino acids, attachment factors, cytokines, growth factors, hormones, antibiotics, therapeutic agents, buffers, etc. The cell culture components and/or conditions may be selected and/or changed during the methods of the present invention to enhance and/or stimulate certain cellular characteristics and/or properties. Examples of seeding methods and cell culturing methods are described in U.S. Pat. Nos. 5,266,480, 5,770,417, 6,537,567, and 6,962,814 and Oberpenning et al. “De novo reconstitution of a functional mammalian urinary bladder by tissue engineering” Nature Biotechnology 17:149-155 (1999), which are incorporated herein by reference in their entirety.

In some embodiments, the hydrogel, hydrogel matrix, and/or neuronal cell culture medium comprises any one or more of the following components: artemin, ascorbic acid, ATP, B-endorphin, BDNF, bovine calf serum, bovine serum albumin, calcitonin gene-related peptide, capsaicin, carrageenan, CCL2, ciliary neurotrophic factor, CX3CL1, CXCL1, CXCL2, D-serine, fetal bovine serum, fluorocitrate, formalin, glial cell line-derived neurotrophic factor, glial fibrillary acid protein, glutamate, IL-1, IL-1α, IL-1β, IL-6, IL-10, IL-12, IL-17, IL-18, insulin, laminin, lipoxins, mac-1-saporin, methionine sulfoximine, minocycline, neuregulin-1, neuroprotectins, neurturin, NGF, nitric oxide, NT-3, NT-4, persephin, platelet lysate, μmX53, Poly-D-lysine (PLL), Poly-L-lysine (PLL), propentofylline, resolvins, SI00 calcium-binding protein B, selenium, substance P, TNF-α, type I-V collagen, and zymosan.

As described herein, the term “optogenetics” refers to a biological technique which involves the use of light to control cells in living tissue, typically neurons, that have been genetically modified to express light-sensitive ion channels. It is a neuromodulation method employed in neuroscience that uses a combination of techniques from optics and genetics to control and monitor the activities of individual neurons in living tissue—even within freely-moving animals—and to precisely measure the effects of those manipulations in real-time. The key reagents used in optogenetics are light-sensitive proteins. Spatially-precise neuronal control is achieved using optogenetic actuators like channelrhodopsin, halorhodopsin, and archaerhodopsin, while temporally-precise recordings can be made with the help of optogenetic sensors for calcium (Aequorin, Cameleon, GCaMP), chloride (Clomeleon) or membrane voltage (Mermaid). In some embodiments, neural cells modified with optogenetic actuators and/or sensors are used in the culture systems described herein.

The term “plastic” refers to biocompatible polymers comprising hydrocarbons. In some embodiments, the plastic is selected from the group consisting of: Polystyrene (PS), polyester, Poly acrylo nitrile (PAN), Poly carbonate (PC), polyvinylpyrrolidone, polybutadiene (PVP), Polyvinyl butyral (PVB), Poly vinyl chloride (PVC), Poly vinyl methyl ether (PVME), poly lactic-co-glycolic acid (PLGA), poly(l-lactic acid), polyester, polycaprolactone (PCL), poly ethylene oxide (PEO), polyaniline (PANI), polyflourenes, polypyrroles (PPY), poly ethylene dioxythiophene (PEDOT), and a mixture of two or any of the foregoing polymers. In some embodiments the composition of the permeable solid support comprises varying degrees of flexibility or flexural modulus. In some embodiments, the permeable solid support comprises a flexural modulus of from about 0.2 to about 20 GP, or Gigapascals. In some embodiments, the permeable solid support comprises a flexural modulus of from about 0.2 to about 20 GP. In some embodiments, the permeable solid support comprises a flexural modulus of from about 0.9 to about 10 GP. In some embodiments, the permeable solid support comprises a flexural modulus of from about 0.2 to about 4 GP. In some embodiments, the permeable solid support comprises a flexural modulus of from about 1.5 to about 10 GP. In some embodiments, the permeable solid support comprises a flexural modulus of from about 0.1 to about 18 GP. In some embodiments, the permeable solid support comprises a flexural modulus of from about 0.01 to about 20 GP.

The term “seeding” as used herein is defined as transferring an amount of cells into an insert. The amount may be defined and may use volume or number of cells as the basis of the defined amount. The cells may be part of a suspension.

The term “permeable solid support” as used herein refers to any substance that is a solid support that is free of or substantially free of cellular toxins and comprises pores. In some embodiments, the permeable solid support comprises one or a combination of polyester, polyvinyl, silica, plastic, glass, and metal. In some embodiments, the permeable solid support comprises pores of a size and shape sufficient to allow diffusion or non-active transport of proteins, nutrients, and gas through the solid substrate in the presence of a cell culture medium. In some embodiments, the pore size is no more than about 10, 9, 8, 7, 6, 5, 4, 3, 2, 1, 0.5, 0.1 microns in diameter. One of ordinary skill could determine how big of a pore size is necessary based upon the contents of the cell culture medium and exposure of cells growing on the permeable solid support in a particular microenvironment. For instance, one of ordinary skill in the art can observe whether any cultured cells in the system or device are viable under conditions with a permeable solid support comprising pores of various diameters. In some embodiments, the permeable solid support comprises a base with a predetermined shape that defines the shape of the exterior and interior surface. In some embodiments, the base comprises one or a combination of polyester, polyvinyl, silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber; and wherein the opening is positioned at one end of the cylinder, in some embodiments, the base comprises one or a plurality of pores of a size and shape sufficient to allow diffusion of protein, nutrients, and oxygen through the solid substrate in the presence of the cell culture medium. In some embodiments, the permeable solid support comprises a polyester base with a pore size of no more than 1 micron in diameter and comprises at least one layer of hydrogel matrix; wherein the hydrogel matrix comprises at least a first cell-impenetrable polymer and at least a first cell-penetrable polymer; the base comprises a predetermined shape around which the first cell-impenetrable polymer and at least a first cell-penetrable polymer physically adhere or chemically bond; wherein the permeable solid support comprises at least one compartment defined at least in part by the shape of an interior surface of the permeable solid support and accessible from a point outside of the permeable solid support by an opening, optionally positioned at one end of the permeable solid support. In embodiments, where the permeable solid support comprises a hollow interior portion defined by at least one interior surface, the cells in suspension or tissue explants may be seeded by placement of cells at or proximate to the opening such that the cells may adhere to at least a portion the interior surface of the permeable solid support prior to growth. The at least one compartment or hollow interior of the permeable solid support allows a containment of the cells in a particular three-dimensional shape defined by the shape of the interior surface of the permeable solid support and encourages directional growth of the cells away from the opening. In the case of neuronal cells, the degree of containment and shape of the at least one compartment are conducive to axon growth from soma positioned within the at least one compartment and at or proximate to the opening, in some embodiments, the permeable solid support is tubular or substantially tubular such that the interior compartment is cylindrical or partially cylindrical in shape. In some embodiments, the permeable solid support comprises one or a plurality of branched tubular interior compartments. In some embodiments, the bifurcating or multiply bifurcating shape of the hollow interior portion of the solids is configured for or allows axons to grow in multiple branched patterns. When and if electrodes are placed at to near the distal end of an axon and at or proximate to a neuronal cell soma, electrophysiological metrics, such as intracellular action potential can be measured within the device or system.

The disclosure relates to an insert comprising a permeable solid support with one or a series of protrusions physically attached or bonded, covalently or non-covalently to the top surface of the permeable solid support. In some embodiments the protrusion is a support ring. The support ring can be affixed to the permeable solid support on its edge in a planar formation defining a cylindrical vessel on the top surface of the permeable solid support. In some embodiments, the height of the edge of the support ring when attached on its edge in a planar orientation is from about 0.1 to about 3 millimeters as measured from the attachment point and continuing to its highest point above the permeable solid support. In some embodiments, the height of the edge of the support ring when attached on its edge in a planar orientation is from about 0.1 to about 2 millimeters as measured from the attachment point and continuing to its highest point above the permeable solid support. In some embodiments, the height of the edge of the support ring when attached on its edge in a planar orientation is from about 0.1 to about 1 millimeter as measured from the attachment point and continuing to its highest point above the permeable solid support. The support ring may be made of any one or plurality of substances including the same substances that make up the permeable solid support.

In some embodiments, the insert comprises a second ring (or culture ring) that can rest coincentrically on top of or around the support ring. The culture ring can be made of any plastic, glass or metal. In some embodiments, the height of the edge of the culture ring when attached on its edge in a planar orientation is from about 0.5 to about 15 millimeters as measured from the attachment point and continuing to its highest point above the permeable solid support. In some embodiments, the height of the edge of the culture ring when attached on its edge in a planar orientation is from about 1 to about 20 millimeters as measured from the attachment point and continuing to its highest point above the permeable solid support. In some embodiments, the height of the edge of the culture ring when attached on its edge in a planar orientation is from about 5 to about 15 millimeters as measured from the attachment point and continuing to its highest point above the permeable solid support. In some embodiments, the culture ring has a lip protruding laterally or substantially laterally, and optionally in parallel, on it top edge when oriented with its longitudinal axis in a vertical or substantially vertical position from with the permeable solid support.

The protrusions may also be in any three-dimensional shape such as a hollow rectangular prism, cylinder such that the sides of such shape define a barrier between the inner and outer surface of the permeable solid support.

Electrodes. In some embodiments, the insert, system and/or adapter disclosed herein comprise one or more electrodes. In some embodiments, the insert, system and/or adapter disclosed herein do not comprise one or more electrodes at or near a position distal from the surface intended for seeding cells. In some embodiments, the one or more electrodes transmit current variation generated by a source of electricity, such as an amplifier operably linked to the electrode via a circuit or series of wires. In some embodiments, the electrodes comprise any conductive material or metal. In some embodiments, the electrodes comprise a carbon scaffold upon which a metal is deposited. In some embodiments, the electrodes comprise a carbon scaffold of carbon nanotubes.

Electrode structures which are suitable for the present disclosure and methods for the production of such structures have already been suggested in array technology for other purposes. In this regard, reference is made to U.S. Pat. No. 6,645,359 and its content is incorporated herein by reference in its entirety. Electrodes or Electrically conductive tracks are created or isolated on a first surface such as the top surface of the permeable solid support. Tracks represent the electrodes of the insert. As used herein, the phrase “electrode set” is a set of at least two electrodes, for example 2 to 200, or 3 to 20, electrodes. These electrodes may, for example, be a working (or measuring) electrode and an auxiliary electrode. In some embodiments, tracks cooperate to form an interdigitated electrode array positioned within the periphery of recesses and leads that extend from the inner region of the insert and between recesses toward the periphery of the permeable solid support or the edge of the adapter. Tracks are constructed from electrically conductive materials. Non-limiting examples of electrically-conductive materials include aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (such as highly doped polycrystalline silicon), silver, tantalum, tin, titanium, tungsten, uranium, vanadium, zinc, zirconium, mixtures thereof, and alloys, oxides, or metallic compounds of these elements. In some embodiments, tracks comprise gold, platinum, palladium, iridium, or alloys of these metals, since such noble metals and their alloys are unreactive in biological systems. In some embodiments, the track is a working electrode made of titanium and/or gold, and track is an auxiliary electrode that is also made of titanium and/or gold and is substantially the same size as the working electrode.

Tracks are isolated from the rest of the electrically conductive surface by laser ablation. Techniques for forming electrodes on a surface using laser ablation are known. Techniques for forming electrodes on a surface using laser ablation are known. See, for example, U.S. patent application Ser. No. 09/411,940, filed Oct. 4, 1999, and entitled “LASER DEFINED FEATURES FOR PATTERNED LAMINATES AND ELECTRODE”, the disclosure of which is expressly incorporated herein by reference. Tracks are preferably created by removing the electrically conductive material from an area extending around the electrodes. Therefore, tracks are isolated from the rest of the electrically-conductive material on a surface by a gap having a width of about 5 nm to about 500 nm, preferably the gap has a width of about 100 nm to about 200 nm. Alternatively, it is appreciated that tracks may be created by laser ablation alone on bottom substrate. Further, tracks may be laminated, screen-printed, or formed by photolithography through a mask such as the mask depicted in FIG. 1. Multi-electrode arrangements are also possible in accordance with this disclosure. For example, it is contemplated that an insert or adapter may be formed that includes 4, 5, 6, 7, 8, 9, 10, 11, 12 or more electrically conductive tracks. It is also appreciated that an alternative three-electrode arrangement is possible where tracks are working electrodes and a third electrode is provided as an auxiliary or reference electrode. It is appreciated that the number of tracks, as well as the spacing between tracks in the insert or adapter may vary in accordance with this disclosure and that a number of arrays may be formed within an insert vessel as will be appreciated by one of skill in the art. in some embodiments, the electrodes are embedded on or attached to the permeable solid support or adapter comprising a plastic and/or paper material. Micro-electrode arrays are structures generally having two electrodes of very small dimensions, typically with each electrode having a common element and electrode elements or micro-electrodes. If “interdigitated” the arrays are arranged in an alternating, finger-like fashion (See, e.g., U.S. Pat. No. 5,670,031). These are a sub-class of micro-electrodes in general. Interdigitated arrays of micro-electrodes, or IDAs, can exhibit desired performance characteristics; for example, due to their small dimensions, IDAs can exhibit excellent signal to noise ratios. Interdigitated arrays have been disposed on non-flexible substrates such as silicon or glass substrates, using integrated circuit photolithography methods. IDAs have been used on non-flexible substrates because IDAs have been considered to offer superior performance properties when used at very small dimensions. At such small dimensions, the surface structure of a substrate (e.g., the flatness or roughness) becomes significant in the performance of the IDA. Because non-flexible substrates, especially silicon, can be processed to an exceptionally smooth, flat, surface, these have been used with IDAs. In some embodiments, the at least one electrode is a component of any IDA disclosed herein. FIG. 3 is an example of an IDA wherein multiple electrodes span a horizontal portion of the bottom surface of the vessel or cavity of hydrogel matrix and are oriented in parallel or substantial parallel fashion over a series of cross-sections of the material. When in electrical communication with an amplifier in a system, any number of electrodes may be independently addressable in the system to measure recordings or electrophysiological metrics at one or a series of positions within the culture.

### Methods

In one embodiment, projection photolithography using a digital micromirror device (DMD) is employed to micro pattern a combination of polyethylene glycol dimethacrylate and Puramatrix hydrogels. This method enables rapid micropatterning of one or more hydrogels directly onto a permeable solid support or insert. Because the photomask never makes contact with the gel materials, multiple hydrogels can rapidly be cured in succession, enabling fabrication of many dozens of gel constructs in an hour, without automation. This approach enables the use of polyethylene glycol (PEG), a mechanically robust, cell growth-restrictive gel, to constrain neurite growth within a biomimetic, growth conducive gel. In some embodiments, this growth-conducive gel may be Puramatrix, agarose, or methacrylated dextran. When embryonic dorsal root ganglion (DRG) explants are grown in this constrained three dimensional environment, axons grow out from the ganglion with high density and fasciculation. The majority of axons appear as small diameter, unmyelinated fibers that grow to lengths approaching 1 μm in 2 to 4 weeks. The structure of this culture model with a dense, highly-parallel, three dimensional neural fiber tract extending out from the ganglion is roughly analogous to peripheral nerve architecture. Its morphology may be assessed using neural morphometry, allowing for clinically-analogous assessment unavailable to traditional cellular assays.

The term “recording” as used herein is defined as measuring the responses of one or more neuronal cells. Such responses may be electro-physiological responses, for example, patch clamp electrophysiological recordings or field potential recordings.

The present disclosure also relates to a method of evaluating the relative degree of toxicity of a first agent as compared to a second agent comprising: (a) culturing one or more neuronal cells and/or one or more tissue explants on any of the devices disclosed herein; (b) exposing a first agent and a second agent to the one or more neuronal cells and/or one or more tissue explants in sequence or in parallel time periods (in sequence if on the same set of cells or in parallel if on a second set of cells—for instance, in a multiplexed system); (c) measuring and/or observing one or more morphometric changes of the one or more neuronal cells and/or one or more tissue explants; and (d) correlating one or more morphometric changes of the one or more neuronal and/or one or more tissue explants cells with the toxicity of the first agent; and (e) correlating one or more morphometric changes of the one or more neuronal and/or one or more tissue explants cells with the toxicity of the second agent; and (f) comparing the toxicities of the first and second agent; and (g) characterizing the first or second agent as more toxic or less toxic than the second agent. In some embodiments, when characterizing the first or second agent as more toxic or less toxic than the second agent, if the morphometric changes induced by the first agent are more severe and indicative of decreased cell viability to a greater extent than the second compound, the first agent is more toxic than the second agent; and, if the morphometric changes induced by the first agent are less severe and/or indicative of increased cell viability as compared to the second compound, then the second agent is more toxic than the first agent. The same characterization can be applied in embodiments in which electrophysiological metrics are observed and/or measured.

In some embodiments, the degree of toxicity is determined by repeating any one or more of the steps provided herein with one or a series of doses or amounts of an agent. Rather than comparing or contrasting the relative toxicities among two different agents, one of skill in the art can this way add varying doses of the same agent to characterize when and at what dose the agent may become toxic to the one or plurality of neurons.

The present disclosure also relates to a method of evaluating the toxicity of an agent comprising: (a) culturing one or more neuronal cells and/or one or more tissue explants on any of the devices disclosed herein; (b) exposing at least one agent to the one or more neuronal cells and/or one or more tissue explants; (c) measuring and/or observing one or more electrophysiological metrics of the one or more neuronal cells and/or one or more tissue explants; and (d) correlating one or more electrophysiological metrics of the one or more neuronal cells and/or one or more tissue explants with the toxicity of the agent, such that, if the electrophysiological metrics are indicative of decreased cell viability, the agent is characterized as toxic and, if the electrophysiological metrics are indicative of unchanged or increased cell viability, the agent is characterized as non-toxic; wherein step (c) optionally comprises and/or observing one or more morphometric changes of the one or more neuronal cells and/or one or more tissue explants; and wherein step (d) optionally comprises correlating one or more morphometric changes of the one or more neuronal cells and/or tissue explants with the toxicity of the agent, such that, if the morphometric changes are indicative of decreased cell viability, the agent is characterized as toxic and, if the morphometric changes are indicative of unchanged or increased cell viability, the agent is characterized as non-toxic.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs such as bacterial antibiotics. In some embodiments, the at least one agent comprises a therapeutically effective amount of an antibody, such as a clinically relevant monoclonal antibody like Tysabri.

In some embodiments, the one or more electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells. In some embodiments, the one or more electrophysiological metrics comprise compound action potential across a tissue explant or across a monolayer of any type of cell or mixture of types of cells. In some embodiments, the one or more electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells. In some embodiments, the one or more electrophysiological metrics comprise compound action potential across a tissue explant.

Any types of cells may be used in the insert or in the disclosed systems. In some embodiments, the cells in the insert are free of neuronal cells. In some embodiments the cells are muscle cells, cardiac cells, endothelial cells, neuronal cells, or any combination thereof. In some embodiments, the cells are in a spheroid structure comprising any cell type or mixture of cell types. In some embodiments, the spheroid comprises one or a plurality of one or combination of immune cells chosen from: a T cell, B cell, macrophage and astrocytes. In some embodiments, the spheroid comprises one or a plurality of one or a combination of stem cells chosen from: an embryonic stem cell, a mesenchymal stem cell, and an induced pluripotent stem cell. In some embodiments, the neuronal cell is derived from a stem cell chosen from: an embryonic stem cell, a mesenchymal stem cell, and an induced pluripotent stem cell. In some embodiments, the spheroid comprises one or a plurality of one or combination of immune cells chosen from: a T cell, B cell, macrophage and astrocytes. In some embodiments, the spheroid comprises one or a plurality of one or a combination of stem cells chosen from: an embryonic stem cell, a mesenchymal stem cell, and an induced pluripotent stem cell. In some embodiments, the neuronal cell is derived from a stem cell chosen from: an embryonic stem cell, a mesenchymal stem cell, and an induced pluripotent stem cell.

In some embodiments, the spheroid has a diameter from about 200 microns to about 700 microns. In some embodiments, the spheroid comprises one or a plurality of neuronal cells and one or a plurality of Schwann cells at a ratio of cell types equal to about 4 neuronal cells for every 1 Schwann cell. In some embodiments, the spheroid comprises one or a plurality of neuronal cells and one or a plurality of astrocytes at a ratio of about 4 neuronal cells for every 1 astrocyte. In some embodiments, the spheroid comprises one or a plurality of neuronal cells and one or a plurality of astrocytes at a ratio of about 1 neuronal cell for every 1 astrocyte. In some embodiments, the spheroid comprises one or a plurality of neuronal cells and one or a plurality of Schwann cells at a ratio of about 10 neuronal cells for every 1 Schwann cell. In some embodiments, the spheroid comprises one or a plurality of neuronal cells and one or a plurality of glial cells at a ratio equal to about four neuronal cells for every 1 glial cell.

In some embodiments, any one or plurality of cells described herein are differentiated from induced pluripotent stem cells. In some embodiments, the spheroid are free of induced pluripotent stem cells and/or immune cells. In some embodiments, the spheroid are free of undifferentiated stem cells.

In some embodiments, the spheroid comprises no less than about 30,000, 35,000, 40,000, 45,000, 50,000, 55,000, 60,000, 65,000, 70,000, 75,000 cells. In some embodiments, the spheroid comprises no less than 75,000 cells.

In some embodiments, the spheroid further comprises one or a plurality of magnetic particles. As used herein, a “spheroid” or “cell spheroid” means any grouping of cells in a three-dimensional shape that generally corresponds to an oval or circle rotated about one of its principal axes, major or minor, and includes three-dimensional egg shapes, oblate and prolate spheroids, spheres, and substantially equivalent shapes. A spheroid of the present invention can have any suitable width, length, thickness, and/or diameter. In some embodiments, a spheroid may have a width, length, thickness, and/or diameter in a range from about 10 μm to about 50,000 μm, or any range therein, such as, but not limited to, from about 10 μm to about 900 μm, about 100 μm to about 700 μm, about 300 μm to about 600 μm, about 400 μm to about 500 μm, about 500 μm to about 1,000 μm, about 600 μm to about 1,000 μm, about 700 μm to about 1,000 μm, about 800 μm to about 1,000 μm, about 900 μm to about 1,000 μm, about 750 μm to about 1,500 μm, about 1,000 μm to about 5,000 μm, about 1,000 μm to about 10,000 μm, about 2,000 to about 50,000 μm, about 25,000 μm to about 40,000 μm, or about 3,000 μm to about 15,000 μm. In some embodiments, a spheroid may have a width, length, thickness, and/or diameter of about 50 μm, 100 μm, 200 μm, 300 μm, 400 μm, 500 μm, 600 μm, 700 μm, 800 μm, 900 μm, 1,000 μm, 5,000 μm, 10,000 μm, 20,000 μm, 30,000 μm, 40,000 μm, or 50,000 μm. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell. In some embodiments, spheroids may be made up of one, two, three or more different cell types, including one or a plurality of neuronal cell types and/or one or a plurality of stem cell types. In some embodiments, the interior core cells may be made up of one, two, three, or more different cell types. In some embodiments, the exterior surface cells may be made up of one, two, three, or more different cell types. In some embodiments, the spheroids comprise at least two types of cells. In some embodiments the spheroids comprise neuronal cells and non-neuronal cells. In some embodiments, the spheroids comprise neuronal cells and astrocytes at a ratio of about 5:1, 4:1, 3:1, 2:1 or 1:1 of neuronal cells to astrocytes. In some embodiments, the spheroids comprise neuronal cells and non-neuronal cells at a ratio of about 5:1, 4:1, 3:1, 2:1 or 1:1. In some embodiments, the spheroids comprise neuronal cells and non-neuronal cells at a ratio of about 1:5: 1:4, 1:3, or 1:2. Any combination of cell types disclosed herein may be used in the above-identified ratios within the spheroids of the disclosure.

Depending on the particular embodiment, groups of cells may be placed according to any suitable shape, geometry, and/or pattern. For example, independent groups of cells may be deposited as spheroids, and the spheroids may be arranged within a three dimensional grid, or any other suitable three dimensional pattern. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures.

The present invention relates to systems comprising the insert and/or adapter. In some embodiments the system comprises a circuit that operably links the electrodes, contact pads and measurement devices (such as voltmeters and ammeters) to a electricity source, such as an amplifier. In some embodiments the insert and/or the adapter are configured for electrical connection to one or more commercially available devices that generate and/or measure voltage drops and supply current across a circuit. In some embodiments, the system comprises a Alpha MED Scientific MED 64 (http://www.med64.com/products/), Axion Biosystems Maestro (https://www.axionbiosystems.com/products), or a Multichannel Systems 2100MEA-System (https://www.multichannelsystems.com/products/mea2100-systems), the contents of such product webpages are incorporated by reference in their entireties. The present disclosure also relates to a system comprising: (i) an insert comprising a hydrogel; (ii) one or a plurality of cells either in suspension or as a component of a tissue explant; (iii) an amplifier comprising a generator for electrical current; (iv) a voltmeter and/or ammeter; (v) at least a first stimulating electrode and at least a first recording electrode; wherein the amplifier, voltmeter and/or ammeter, and electrodes are electrically connected to the each other via a circuit in which electrical current is fed to the at least one stimulating electrode from the amplifier and electrical current is received at the recording electrode and fed to the voltmeter and/or ammeter; wherein the stimulating electrode is positioned at or proximate to one or a plurality of cells at one end of a cavity and the recording electrode is positioned at a predetermined distance distal to the first electrode, such that an electrical field is established across the insert. In some embodiments the system comprises an insert resting within a multiwall chamber of a multiwall tissue culture plate. In some embodiments, the system comprises any of the commercially disclosed amplifier containing devices; an insert positioned within the adapter and electrically linked to the amplifier via a circuit.

Systems that measure physiological metrics are described and known in PCT Application No. PCT/US2015/050061 and currently pending U.S. Provisional Application Ser. No. 62/594,525, filed Dec. 4, 2017, the content of both of which are incorporated by reference in their entireties.

### Methods

Once seeded in the inner region of the insert, whether on hydrogel or without hydrogel and directly on the inner region of the permeable solid support, any suitable physiological response of the cells or the spheroid may be determined, evaluated, measured, and/or identified in a method of the present disclosure. In some embodiments, 1, 2, 3, 4, or more physiological response(s) of the cells or spheroid may be determined, evaluated, measured, and/or identified in a method of the present disclosure. In some embodiments, the physiological response of the cell or spheroid may be a change in morphology of the cell or the spheroid. The method may comprise determining a change in morphology of the cell or the spheroid, which may include estimating at least one morphology parameter prior to contacting the spheroid with an agent, such as a chemical and/or biological compound, estimating the at least one morphology parameter after contacting the cell or the spheroid with the agent, and calculating the difference between the at least one morphology parameter prior to and after contacting the spheroid with the agent to provide the change in morphology for the spheroid. In some embodiments, the physiological response of the cell or the spheroid may be the cells or spheroid shrinking or swelling in response to contact with an agent. Morphology of the cells or spheroid may be determined using any methods known to those of skill in the art, such as, but not limited to, quantifying eccentricity and/or cross sectional area.

In some embodiments, the physiological response of the cells or spheroid may be a change in volume of the cells or the spheroid. The method may comprise determining a change in volume for the cells or spheroid, which may include estimating a first volume prior to contacting the cell or spheroid with an agent, estimating a second volume after contacting the cells or spheroid with the agent, and calculating the difference between the first volume and the second volume to provide the change in volume for the cells or spheroid. In some embodiments, the physiological response of the spheroid may be the cells or spheroid shrinking or swelling in response to contact with an agent.

The present disclosure also relates to method of measuring the amount or degree of myelination or demyelination of one or more axons of one or a plurality of neuronal cells and/or one or a plurality of tissue explants, said method comprising: (a) culturing one or more neuronal cells and/or one or a plurality of tissue explants on any of the devices disclosed herein for a time and under conditions sufficient to grow at least one axon; (b) measuring and/or observing one or more morphometric changes of the one or more neuronal cells and/or one or more tissue explants; and (c) correlating one or more morphometric changes of the one or more neuronal and/or one or more tissue explants cells with a quantitative or qualitative change of myelination of the neuronal cells or tissue explants.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants on any of the devices disclosed herein for a time and under conditions sufficient to grow at least one axon; (b) measuring and/or observing one or more electrophysiological metrics of the one or more neuronal cells and/or one or more tissue explants; and (c) correlating one or more electrophysiological metrics of the one or more neuronal and/or one or more tissue explants cells with a quantitative or qualitative change of myelination of the neuronal cells or tissue explants; wherein step (b) optionally comprises and/or observing one or more morphometric changes of the one or more neuronal cells and/or one or more tissue explants; and wherein step (c) optionally comprises correlating one or more morphometric changes of the one or more neuronal cells and/or tissue explants with the quantitative or qualitative change of myelination of the neuronal cells or tissue explants.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants on any of the devices disclosed herein for a time and under conditions sufficient to grow at least one axon; and (b) detecting the amount of myelination on one or a plurality of axons of the one or more neuronal cells and/or one or more tissue explants.

In some embodiments, the step of detecting the amount of myelination on one or a plurality of axons of the one or more neuronal cells and/or one or more tissue explants comprises exposing the cells to an antibody that binds to myelin.

In some embodiments, the method further comprises (i) exposing one or a plurality of neuronal cells and/or one or a plurality of tissue explants to at least one agent after steps (a) and (b); (ii) measuring and/or observing one or more electrophysiological metrics, measuring and/or observing one or more morphometric changes and/or detecting the quantitative amount of myelin from the one or a plurality of neuronal cells and/or one or a plurality of tissue explants; (iii) calculating a change of measurements, observations and/or quantitative amount of myelin from the one or a plurality of neuronal cells and/or the one or a plurality of tissue explants in the presence and absence of the agent; and (iv) correlating the change of measurements, observations and/or quantitative amount of myelin from the one or a plurality of neuronal cells and/or the one or a plurality of tissue explants to the presence or absence of the agent.

In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs.

In some embodiments, the one or more electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells. In some embodiments, wherein the one or more electrophysiological metrics comprise compound action potential across a tissue explant.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants on any of the devices disclosed herein for a time and under conditions sufficient to grow at least one axon; and (b) inducing a compound action potential in such one or more neuronal cells and/or one or more tissue explants; (c) measuring the compound action potential; and (d) quantifying the levels of myelination of such one or more neuronal cells based on the compound action potential. In some embodiments, the method further comprises exposing the one or more neuronal cells and/or one or a plurality of tissue explants to an agent. In some embodiments, the at least one agent comprises at least one environmental or industrial pollutant.

In some embodiments, the at least one agent comprises one or a combination of small chemical compounds chosen from: chematherapeutics, analgesics, cardiovascular modulators, cholesterol level modulators, neuroprotectants, neuromodulators, immunomodulators, anti-inflammatories, and anti-microbial drugs.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine(DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide(Oral dacarbazine), Teniposide, Tioguanine (formerly Thioguanine), Topotecan, Tretinoin, Valrubicin, Vemurafenib, Vinblastine Vincristine, Vindesine, Vinorelbine, Vismodegib, and Vorinostat.

In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, Scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GAB A, calcium, acetylcholine, epinephrine, norepinephrine, and serotonin.

In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate.

In some embodiments, the at least one agent comprises one or a combination of antimicrobials chosen from: antibacterials, antifungals, antivirals, antiparasitics, heat, radiation, and ozone.

In some embodiments, the method further comprises measuring one or a plurality of electrophysiological metrics other than compound action potential chosen from one or a combination of: electrical conduction velocity, individual action potential, amplitude of the wave associated with passage of an electrical impulse along a membrane of one or a plurality of neuronal cells and/or tissue explants, a width of an electrical impulses along a membrane of one or a plurality of neuronal cells and/or tissue explants, latency of the electrical impulse along a membrane of one or a plurality of neuronal cells and/or tissue explants, and envelope of the electrical impulse along a membrane of one or a plurality of neuronal cells and/or tissue explants. In some embodiments, the method further comprises measuring one or more morphometric changes associated with the one or more neuronal cells and/or the one or plurality of tissue explants.

The present disclosure also relates to a method of inducing growth of one or a plurality of neuronal cells on any of the devices disclosed herein, said method comprising: (a) seeding one or a plurality of isolated Schwann cells within the hydrogel matrix on the permeable solid support; (b) seeding one or a plurality of isolated neuronal cells in suspension or isolated neuronal cells in an explant to the device; (c) introducing a cell culture medium into the vessel with a volume sufficient to cover the cells; wherein the hydrogel matrix comprises a first cell-impenetrable polymer and a first cell-penetrable polymer.

In some embodiments, the method further comprises positioning at least one electrode at either end or both ends of the permeable solid support, such that the electrodes can be used to stimulate or record action potentials (APs) and or compound action potentials (cAPs) allowing measurement of AP/cAP propagation.

In some embodiments, the electrode or electrodes are positioned at or distal to the soma of dorsal root ganglion (DRG) neurons such that the electrodes create a voltage difference between two points of the neurites/axons to evoke a propagating AP/cAP.

The present disclosure also relates to a method of assessing the response of the neuronal cells on the insert following introduction of one or more stimuli to the one or more neuronal cells; and measuring AP or cAP responses from the one or more neuronal cells to the one or more stimuli using local field potential (LFP) or other recording methods.

In some embodiments, the permeable solid support comprises an exterior surface and an interior surface, such solid substrate comprising at least one portion in a cylindrical or substantially cylindrical shape and at least one hollow interior defined at its edge by at least one portion of the interior surface; said interior surface comprising one or a plurality of pores from about 0.1 microns to about 3.0 microns in diameter, wherein the hollow interior of the solid substrate is accessible from a point exterior to the permeable solid support through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with at least one of the first cell-impenetrable polymer or the first cell-penetrable polymer, and wherein the second portion of the at least on hollow interior is in fluid communication with the first portion such that axons are capable of growth from the one or plurality of neuronal cells and/or the one or plurality of tissue explants into the second interior portion of the hollow interior.

In some embodiments, the method further comprises contacting the one or plurality of neuronal cells with at least one agent. In some embodiments, the at least one agent is one or a plurality of stem cells or modified T cells. In some embodiments, the modified T cells express chimeric antigen receptors specific for a cancer cell. In some embodiments, the cell culture medium comprises one or a combination of: laminin, insulin, transferrin, selenium, BSA, FBS, ascorbic acid, type I collagen, and type III collagen.

The present disclosure also relates to a method of detecting and/or quantifying neuronal cell growth comprising: (a) quantifying one or a plurality of neuronal cells; (b) culturing the one or more neuronal cells on any of the devices disclosed herein; and (c) calculating the number of neuronal cells in the composition after culturing for a time period sufficient to allow growth of the one or plurality of cells. In some embodiments, step (c) comprises detecting an internal and/or external recording of such one or more neuronal cells after culturing one or more neuronal cells and correlating the recording with a measurement of the same recording corresponding to a known or control number of cells.

In some embodiments, the method further comprises contacting the one or more neuronal cells to one or more agents. In some embodiments, the method further comprises: (i) measuring an intracellular and/or extracellular recording before and after the step of contacting the one or more neuronal cells to the one or more agents; and (ii) correlating the difference in the recordings before contacting the one or more neuronal cells to the one or more agents to the recording after contacting the one or more neuronal cells to the one or more agents to a change in cell number.

The present disclosure also relates to a method of detecting or quantifying of axon degeneration of one or a plurality of neuronal cells comprising: (a) seeding one or a plurality of neuronal cells on any of the devices disclosed herein; (b) culturing the one or plurality of neuronal cells for a time period and under conditions sufficient to grow at least one or a plurality of axons from the one or plurality of neuronal cells, (c) quantifying the number or density of axons grown from the neuronal cells; (d) contacting the one or plurality of neuronal cells to one or a plurality of agents; (e) quantifying the number and/or the density of the axons grown from neuronal cells after contacting the one or plurality of cells to one or a plurality of agents; and (f) calculating a difference in the number or density of axons in culture in the presence or absence of the agent.

In some embodiments, the step of the one or plurality of axons and/or the density of the axons grown from neuronal cells comprises staining the one or plurality of a neuronal cells with a dye, fluorophore, or labeled antibody.

In some embodiments, steps (c), (e), and/or (f) are performed via microscopy or digital imaging.

In some embodiments, steps (c) and (e) comprise taking measurements comprises from a portion of one or plurality of axons proximate to one or a plurality soma and taking measurements from a portion of one or plurality of axons distal to one or a plurality soma.

In some embodiments, the difference in the number or density of axons in culture in the presence or absence of the agent is the difference between a portion of the axon or axons proximate to cell bodies of the one or plurality of neuronal cells and a portion of the axons distal from the cell bodies of the one or plurality of neuronal cells.

In some embodiments, taking measurements comprises measuring any one of or combination of: morphometric metrics or electrophysiological metrics and wherein the step of calculating a difference in the number or density of axons in culture comprises correlating any one or combination of measurements to the number or density of axons. In some embodiments, taking measurements comprises measuring any one of or combination of electrophysiological metrics and wherein the step of calculating a difference in the number or density of axons in culture comprises correlating any one or combination of electrophysiological metrics to the number or density of axons.

In some embodiments, the method further comprises (g) correlating the neurodegenerative effect of an agent to electrophysiological metrics taken in steps (c) and (e).

The present disclosure also relates to method of measuring intracellular or extracellular recordings comprising: (a) culturing one or a plurality of neuronal cells on any of the devices disclosed herein; (b) applying a voltage potential across the one or a plurality of neuronal cells; and (c) measuring one or a plurality of electrophysiological metrics from the one or a plurality of neuronal cells. In some embodiments, the one or a plurality of electrophysiological metrics other are chosen from one or a combination of: electrical conduction velocity, intracellular action potential, compound action potential, amplitude of the wave associated with passage of an electrical impulse along a membrane of one or a plurality of neuronal cells and/or tissue explants, a width of an electrical impulses along a membrane of one or a plurality of neuronal cells and/or tissue explants, latency of the electrical impulse along a membrane of one or a plurality of neuronal cells and/or tissue explants, and envelope of the electrical impulse along a membrane of one or a plurality of neuronal cells and/or tissue explants.

The present disclosure also relates to a method of measuring or quantifying any neuroprotective effect of an agent comprising: (a) culturing one or a plurality of neuronal cells or tissue explants on any of the devices disclosed herein in the presence and absence of the agent; (b) applying a voltage potential across the one or a plurality of neuronal cells or tissue explants in the presence and absence of the agent; (c) measuring one or a plurality of electrophysiological metrics from the one or plurality of neuronal cells or tissue explants in the presence and absence of the agent; and (d) correlating the difference in one or a plurality of electrophysiological metrics through the one or plurality of neuronal cells or tissue explants to the neuroprotective effect of the agent, such that a decline in electrophysiological metrics in the presence of the agent as compared to the electrophysiological metrics measured in the absence of the agent is indicative of a poor neuroprotective effect, and no change or an incline of electrophysiological metrics in the presence of the agent as compared to the electrophysiological metrics measured in the absence of the agent is indicative of the agent conferring a neuroprotective effect.

The present disclosure relates to a method of measuring or quantifying any neuromodulatory effect of an agent comprising: (a) culturing one or a plurality of neuronal cells or tissue explants on any of the devices disclosed herein in the presence and absence of the agent; (b) applying a voltage potential across the one or a plurality of neuronal cells or tissue explants in the presence and absence of the agent; (c) measuring one or a plurality of electrophysiological metrics from the one or plurality of neuronal cells or tissue explants in the presence and absence of the agent; and (d) correlating the difference in one or a plurality of electrophysiological metrics through the one or plurality of neuronal cells or tissue explants to the neuromodulatory effect of the agent, such that a change in electrophysiological metrics in the presence of the agent as compared to the electrophysiological metrics measured in the absence of the agent is indicative of a neuromodulatory effect, and no change of electrophysiological metrics in the presence of the agent as compared to the electrophysiological metrics measured in the absence of the agent is indicative of the agent not conferring a neuromodulatory effect.

The present disclosure also relates to a method of detecting or quantifying myelination or demyelination of an axon in vitro comprising: (a) culturing one or a plurality of neuronal cells on any of the devices disclosed herein for a time and under conditions sufficient for the one or a plurality of neuronal cells to row one or a plurality of axons; (b) applying a voltage potential across the one or a plurality of neuronal cells; and (c) measuring the field potential or compound action potential through the one or plurality of neuronal cells; (d) calculating the conduction velocity through the one or a plurality of neuronal cells; and (e) correlating the one or plurality of values or conduction velocity with the amount of myelination of one or a plurality of axons.

The following examples are meant to be non-limiting examples of how to make and use the embodiments disclosed in this application. Any publications disclosed in the examples or the body of the specification are incorporated by reference in their entireties.

### EXAMPLES

### Example 1: Design and Manufacturing

### Cell Culture Inserts and Design Factors

Two design factors guided the development of the custom MEA platform in this work. The first was the design of the cell culture inserts used in our lab for culture of our neurite constructs. These cell culture inserts are Corning Transwell®-Clear inserts, each with a 24 mm-diameter polyester membrane as the bottom substrate, with 0.4 μm pores [41]. These pores are necessary for diffusion of cell culture media or other solutions through the membrane, and can become closed upon excess application of heat.

Six of these cell culture inserts are designed to sit in a 6-well plate, suspended from the bottom of the wells by contact of the upper ring. This allows for solution, typically cell culture media, to be placed below the insert, so that it may diffuse up and into tissues, or in our case, neurite constructs. The cell culture inserts are designed in such a way that around the sides, three holes are present, distributed evenly such that there is a 120° angle between each of them. These holes are typically used for accessing the solution placed below the insert.

In order to design a system in which electrodes can interface with other equipment, there must be a way to ensure that the two fit together. For example, in a commercial MEA system, this corresponds to the edge connectors of the MEA matching up with connectors in the interfacing equipment. In order to provide a similar interface for our custom MEA on cell culture inserts, the solution access holes mentioned above prove to be essential for maintaining orientation of the elements involved.

The second design factor was the use of pressure-based contact to form a continuous electrical connection. This is a ubiquitous approach to creating an electrical connection, and can be seen in use in mostly static applications, such as USB ports, and in constantly moving applications, such as brushed direct-current motors. This approach to electrical contact proves to be rapid and facile, and so is incorporated in this work as a method of interfacing.

### Electron Beam Physical Vapor Deposition

Physical vapor deposition (PVD) refers to a number of processes in which material is vaporized into atoms or molecules from a source, and condensed onto a substrate [44]. These processes are used to create films and coatings, or multilayer composites thereof. It has been shown that it is possible to adapt vacuum evaporation PVD processes to create flexible electrodes that can be applied for electrophysiology [45], and so the techniques is applied in this work for custom MEA production.

Two common PVD processes are sputter deposition and vacuum evaporation. Sputter deposition works by energizing the material source to be sputtered onto the substrate with plasma, ejecting material towards the substrate [44]. Sputtering typically involves a short distance between the source and substrate [44].

Vacuum evaporation works by evacuating the chamber containing the source and substrate, and energizing the source, typically with a high energy electron beam, generally referred to as an e-beam [44]. This thermally evaporates the source material, which travels to the substrate by line-of sight. E-beam vacuum evaporation typically involves a relatively long distance between the source and substrate, to minimize heat transfer to the substrate [44].

### Additive Manufacturing

Additive manufacturing refers to processes in which something is manufactured through addition of material to form the item, as opposed to starting with a mass of material and removing material from the item, which corresponds with subtractive manufacturing. Frequently, additive manufacturing refers to 3D printing, a set of technologies for creating real-world models of digitally created parts, that have seen active development since 1984 [46]. By being able to go directly from a digital model to a physical object, 3D printing greatly facilitates the production of prototype apparatuses.

The 3D printing method employed for creating components in this work is known as fused filament fabrication (FFF). In this process, a thermoplastic filament is fed from a spool by a motorized feed mechanism, which pushes it through a heated extruder, melting it. The three motorized axes of the 3D printer then move either the extruder or the build plate, depositing thermoplastic in desired locations for a given layer of the component being fabricated. The vertical axis then moves the extruder further from the build plate, and the process is repeated for the next layer, the thermoplastic of the new layer being fused with that of the lower layer [47].

Two common filaments used for 3D printing are acrylonitrile butadiene styrene (ABS) and polylactic acid (PLA). ABS has a higher glass transition temperature than PLA [48], and so is less prone to deformation under heat load. ABS also is somewhat more flexible, and less brittle, than PLA.

While FFF 3D printing offers simplicity in the production of prototype components, there are disadvantages to the technique. Readily available consumer-grade printers generally are incapable of printing metals, limiting the uses of parts produced by these printers. Since FFF technology relies on manipulating molten material, it also can be prone to dimensional inaccuracies.

### Subtractive Manufacturing

Subtractive manufacturing refers to processes that remove material from an object to produce the desired part. Milling is a common method for subtracting manufacturing, in which a turning tool performs cutting and drilling operation on a piece of source material. Milling is typically done with a machine that, like the extruder of a 3D printer, has a toolhead that is moved by three axes. Modern milling processes often include computer numerical control (CNC) technology, allowing the process to be automated with motorized axes [49]. This level of control results in milled parts being very accurate to the desired part dimensions.

### Summary and Objective

Previous work in our lab has developed a robust platform for investigating peripheral sensory nervous tissue, our hydrogel neurite constructs. This platform gives the cost and relative simplicity advantages of an in vitro neural model, while maintaining characteristics more similar to in vivo nervous tissues. Electrophysiology is a key method for using these constructs as a platform for studying pharmaceutical or pathological effects on nervous tissue. However, the present technique for conducting electrophysiology, electrode-based field potential recording, is inconsistent and would be difficult to automate, limiting the platform's application to large studies in the future.

The objective of this work was to find a rapid, automatable way to perform electrophysiology on hydrogel neurite constructs. This would eliminate the remaining major barrier for large-scale and rapid use of our constructs in the study of nervous pathologies and pharmaceutical responses. Finding a solution for this remaining problem required mitigating the issues present in the current field potential recording technique. Table 1 below summarizes the design problems of probe-based field potential recordings addressed in creation of custom rapid electrophysiology platform. Problems and candidate solutions italicized are complicating factors introduced by a commercial MEA system.

TABLE 1

| Design Problem | Candidate Solutions |
|---|---|
| Constructs have to be physically cut | MEA |
| out of cell culture inserts | |
| Probe placement time-consuming | Hybrid Conductive Hydrogels, |
| | MEA |
| Probe placement inconsistent, nerve | Hybrid Conductive Hydrogels, |
| conduction velocity difficult to establish | MEA |
| High cost | Fabricate custom MEAs |
| Appropriate number of electrodes | Customize MEA design |
| and pattern for neurite construct | |
| Porous membrane | Use existing cell culture inserts |
| | as substrate for custom MEAs |

In order to address the issues present in field potential recordings, several candidate solutions were investigated, including hybrid conductive hydrogels and commercial MEAs. The general design of commercial multielectrode arrays was found to be likely to resolve the issues present in our current electrophysiology technique, however was also shown to be poor fit for our constructs due to further complicating factors with their design. Therefore, a custom platform for rapid electrophysiology based on existing cell culture inserts was developed, incorporating the advantages of commercial MEAs, while designed to avoid the associated complicating factors.

### Electrochemical Impedance Spectroscopy (EIS) Testing Rig Fabrication

Two-point EIS testing rigs were fabricated from high-density polyethylene plastic, copper rods, and nylon fasteners using a band saw and a drill press. The two copper rods in each testing rig were aligned to have 161 mm² of parallel surface area, separated by an average of 0.771 mm, and measured to have a very low capacitive contribution to impedance, ˜2 pF.

### Hydrogel Solution Preparation for EIS

Hydrogel samples for Electrochemical Impedance Spectroscopy (EIS) were prepared as to have as similar as possible composition to the gels that would be formed in hydrogel constructs employing them. PEG solutions were prepared with 1.00 g of 1000 MW PEG, 0.050 g Irgacure® 2959 (BASF), and 10.0 mL phosphate buffered solution (PBS). 8% HP solutions were prepared with 0.040 g of 32% methacrylated HP (Me-HP), 0.010 g Irgacure® 2959, 28.8 μL n-vinylpyrrolidone (NVP), and 0.471 mL PBS. 4% HA solutions were prepared similarly, with 0.020 g of 32% methacrylated HA (Me-HA) in place of Me-HP. Ammonium Persulfate (APS) solutions were prepared with 0.100 g APS and 1.00 mL PBS, and pyrrole (Py) solutions were prepared with 0.100 mL Py and 1.00 mL PBS. All solutions were thoroughly mixed with a vortex mixer prior to use.

### Hydrogel Sample Preparation

For all gel types (HA, HA-Ppy, HP, HP-Ppy, and PEG), a glass slide was prepared with a 70% EtOH wash, followed by an application of Rain-X. A stainless steel washer with an inner diameter of 9.70 mm was placed onto the slide, after being washed and coated in the same fashion as the glass slide. 150 μL of gel solution, HA, HP or PEG, was then added to the center of the washer, and a circular pattern gelated using the DMD with UV light applications of 60 s for HA and HP, and 38 s for PEG. The UV light, upon passage through the DMD and to the substrate, has wavelengths in the range of 375-409 nm, and a surface power density of 85 mW/cm²[50]. The excess fluid was then removed with a KimWipe® (Kimberly-Clark). For the HA-Ppy and HP-Ppy samples, 150 μL, of APS solution was added into the washer (with the HP gel inside) for 60 s, and then removed. 150 μL of PBS was then applied for 10 s and removed, and then 150 μL of Py solution was added, and left until full color change to black was witnessed, about 60 s. The excess Py solution was then removed, and three 10 s 150 μL, PBS washes were conducted.

### EIS Experiment

The copper contacts of the EIS testing rigs were polished with metal polishing paper, and samples were transferred from the glass slide on which they were formed to one contact with a razor blade. The other contact was then placed on top of the sample, and the testing rig was screwed tightly together with the nylon fasteners, securing the sample in place in the process. The testing rigs were then connected to an Agilent 4294A Precision Impedance Analyzer (Agilent Technologies), and subjected to a 500 mV, 100 Hz-1 MHz logarithmic frequency sweep. Impedance and phase information was obtained for each frequency tested.

After impedance testing, the accurate separation of the copper plates was gathered by measuring the open sides of the testing rig with digital calipers, and establishing the average. The diameter of the sample was established by visually lining up the calipers with the visible edges of the sample from both open sides of the testing rig, and averaging the results. Obtained impedance data points were decomposed into resistance using the measured phase angle and impedance at that frequency, as resistance=(impedance) sin(phase angle). Using the sample size information, the resistance was that normalized to obtain the material resistivity, as resistivity=resistance (ArealLength). Resistivity for each sample was tabulated at low frequency (100 Hz) and high frequency (1 MHz). Phase angle was also tabulated for each sample at low frequency. One-way analysis of variance (ANOVA) tests were performed individually for each of the low and high frequency domains of resistivity, and for the low frequency domain of phase angle. Bonferroni post-hoc tests were used to compare the means of each data set where significance was established by ANOVA. Statistics and figures were produced using GraphPad Prism (GraphPad Software, Inc.).

### Custom Multielectrode Arrays (MEAs)

Early attempts at fabricating custom MEAs used sputter deposition. This technique was found to be non-ideal as the permeability of the insert membranes was compromised after being subjected to the deposition procedure, confirmed by applying fluid on top of the insert membrane and seeing no fluid permeate through after an extended period of time. This finding led to the selection of vacuum evaporation PVD, as this process subjected less heat on the insert membrane. Early results using the e-beam vacuum evaporation device showed that insert membranes were not impacted as severely by the process as they were by sputter deposition. This was confirmed by observed fluid permeation through membranes subjected to the technique, and SEM imaging confirming that membrane pores remained present.

### Design of Snap-In Masks for Electron Beam PVD

In order to deposit metal into the desired pattern on the cell culture insert membrane using the e-beam, masks were developed to prevent metal from reaching areas of the membrane where it was not desired. These “snap-in” masks were designed so that they had three stalks oriented at 120° that snapped into the solution access holes of the cell culture inserts. This allowed the masks to stay in place regardless of the orientation of the insert, which was necessary as the e-beam deposits upwards, requiring the inserts to be oriented upside-down when loaded inside. The snap-in stalks also ensured that the mask pattern at the bottom would always be in the same orientation relative to the solution access holes.

Early designs of the snap-in masks were entirely 3D printed. This proved to be and insufficient technique, as the dimensional accuracy of FFF was not able to create the desired small, precise electrode patterns required. The design was thus modified so that the electrode pattern holes in the mask bottom would be CNC milled out of a mask blank 3D printed out of ABS. This also gave an unsatisfactory result, as small filaments of plastic remained attached to the mask holes that would interfere with the deposition of continuous electrodes.

The final mask design mitigated the issue of the remaining plastic filaments by separating the snap stalks and mask details into two different components: a stalk component made of ABS plastic, as to be flexible for facile snapping in and out of inserts; and mask component made of copper metal, as to have a cleaner milled result. Copper was selected as it is has been shown to be an effective mask material for vacuum evaporation processes[45]. Components were designed in SolidWorks® (Dassault Systemes) computer-aided design (CAD) package.

The electrode pattern in the final mask design was decided upon as it is generally similar to the probed electrode configuration previously used for neurite constructs (FIG. 1). Electrodes were designed to be 500 μm in size, as this was the smallest size at which the mask holes could practically be milled. The pattern allows for two neurite constructs per insert, each with a recording electrode, and a stimulating electrode. The ground electrode was placed to serve both as a large ground reference, and to act as the ground half of a bipolar stimulating electrode for both inserts, emulating a probe-type bipolar stimulating electrode. The electrode placement was made to match that of the probes for retrograde electrophysiology, with the stimulus location distal, and the recording location proximal, to the DRG body. Each electrode had a round mating pad added to its end, for interfacing with the custom electrophysiology rig.

### Fabrication of Snap-in Masks

Six snap-in masks of the final design were fabricated. Stalk components were exported as STL files and imported into Cura LulzBot® Edition (Aleph Objects, Inc.) and sliced using standard settings, without support material or bed adhesion, for the Ultimaker 2, and GCODE files exported and copied onto a secure digital (SD) card for printing. Stalk components were individually printed out of grey ABS on an Ultimaker 2 (Ultimaker B.V.) FFF 3D printer with default settings for ABS. Bed adhesion was accomplished by brushing a thin layer of ABS-acetone mixture onto the glass platen, and allowing it to dry, prior to beginning the print. Completed components were removed from the platen with a razor blade, and excess plastic was removed with a pocket knife. The holes in the stalk components for connecting to the bottom components were drilled out with a 1 mm drill bit using a handheld drill in order to ensure that the pegs of the bottom components would fit.

The metal mask bottom components were prepared for milling by importing the SLDPRT part file prepared in SolidWorks, and performing computer-aided machining (CAM) in Autodesk Fusion 360™ (Autodesk Inc.). Milling operations were defined based on the dimensions of the 0.080 in thick copper stock material, the target part, and the square end mills used: a 5/64 in diameter square-ended two-flute end mill used for the larger cutting operations and cutting the final parts out, and a 0.018 in diameter square-ended two-flute end mill for milling mask details. Material feedrates for the milling operations were calculated based on the sizes of these tools, the 10000 RPM top spindle speed of the CNC milling machine, and the copper material, using FSWizard:Online (Eldar Gerfanov).

NC g-code files were exported from the CAM package and loaded into FlashCut CNC (FlashCut CNC) CNC control software on a dedicated computer. Copper stock material (McMaster-Carr) was cut with a bandsaw into strips, and drill-pressed with holes to allow fixturing to the mill with 0.25 in bolts. Sacrifice plywood was laser cut to the same size and fixtured below each copper strip. Three NC files were prepared, one defining the large preliminary cuts, one for the mask details, and a final one to cut the masks from the stock material. Each file was run on all six masks prior to moving on to the next one. An oil-based cutting fluid was applied during cutting operations for chip clearance, cooling, and lubricating purposes. Finished mask bottoms were rinsed with water to remove copper chips, and deburred with a pocket knife.

### E-Beam Mounting Plates for Cell Culture Inserts

In order to quickly and repeatedly place sets of 6 cell culture inserts into the e-beam, a custom set of mounting plates were designed in SolidWorks to attach to the existing substrate mounting plate in the e-beam. These mounting plates were designed to orient the bottoms of the inserts so that they would be radially distributed around the center axis of the existing mounting plate. The custom bottom plate was designed to affix to the existing plate first, then the top plate second, after placing the inserts. 24 mm diameter pieces of 0.125 in thick open-cell, adhesive-backed polyurethane foam were laser cut and added to each of the insert locations on the bottom plate. These foam pieces were added to push the insert membranes up against the snap-in mask bottoms, eliminating any potential gap, thus providing sharper-edged resultant electrodes from e-beam deposition.

The mounting plates were exported as STL files from SolidWorks and imported into Cura LulzBot Edition and sliced using standard settings, with support material, for the LulzBot® TAZ 5 (Aleph Objects, Inc.) FFF 3D printer, and GCODE files exported and copied onto a secure digital (SD) card for printing. The plates were 3D printed out of PLA, and had excess plastic and support material removed with a pocket knife.

### Custom Multielectrode Array Fabrication Procedure

Each e-beam electrode fabrication was done on one new set of 24 mm diameter cell culture inserts, each set consisting of one 6-well plate with 6 corresponding cell culture inserts. Sterile protocol was maintained as much as possible during the preparation of the custom multielectrode arrays. An unopened set of inserts was disinfected with 70% ethanol and brought into the sterile hood. Each component of the 6 snap-in masks was disinfected, brought in, and allowed to dry, as well as a roll of Fisherbrand™ (Fisher Scientific) tape. The snap-ins were then assembled by aligning and inserting the pegs of the bottom component into the corresponding holes in the stalk component. The set of inserts was opened, and each snap-in was applied to an insert. The plate of inserts, with the snap-ins, was then returned to the original sterile packaging, and the packaging was closed again using the tape.

The resealed insert package, tape, custom mounting plates, and ethanol were then carried into the cleanroom, following the appropriate gowning and entry procedures. The e-beam vacuum evaporation PVD device, a Nexdep PVD (Angstrom Engineering), was then opened and the substrate mounting plate removed. The substrate plate and custom plates were wiped down with a cleaning cloth wetted with ethanol, and the center of the bottom custom plate was attached to the center of the substrate plate with a screw. The insert package was then reopened, and the inserts, with the snap-in masks attached, were placed onto the bottom plate. The top plate was then affixed on top with four additional screws, taking care to ensure that the inserts and snap-ins remained in place. Tape was then added to cover all exposed portions of the inserts visible from above.

The completely populated substrate mounting plate was then replaced in the e-beam chamber in its suspended orientation, and the chamber sealed. The chamber was then evacuated for approximately four hours, to a pressure of 1E-7 torr. Once evacuated, a base adhesion layer of titanium was deposited, 5 nm thick, at a rate of 0.3 A/s, followed by the main conducting layer of gold, 45 nm thick, at a rate of 0.5 A/s. These deposition rates were somewhat conservative, as to not apply excess heat to the insert membranes. During the deposition processes, the substrate plate was rotated at approximately 30 rpm, to ensure even deposition across all 6 inserts.

After deposition, the e-beam chamber was returned to atmospheric pressure and opened, and the substrate mounting plate removed. The taping was removed, the top and bottom mounting plates unscrewed from the substrate plate, the snap-in masks removed from the inserts, and the inserts returned to their 6-well plate. The 6-well plate was then replaced in its package, and again sealed with tape. The inserts were then returned to the sterile hood, where each insert had 2 mL of wash solution added in the well below the insert, and another 2 mL added on top of the membrane surface. This wash step was included to remove any potentially remaining metal particulates, and was prepared from 49 mL of PBS and 1 mL of antibiotic-antimycotic (anti-anti) (Gibco). After application of wash solution, the inserts were moved to the incubator, where they were left for at least 24 hours prior to use.

### Custom Electrophysiology Rig Design

The custom electrophysiology rig designed in this work forms the second half of the custom electrophysiology platform, the first being custom multielectrode arrays. The rig was designed so that cell culture inserts with patterned electrodes could very quickly have a continuous electrical connection formed between the ends of the electrodes, directly in contact with neurite constructs, and stimulating and recording electrophysiology equipment. Over more than ten design iterations, a rig design was finalized that achieved this goal. The final rig design consisted of six components: three 3D printed main assembly components, two circuit boards, and a spring (FIG. 5).

The three 3D printed components, those being the base, the plunger, and the main assembly, were designed to be easily assembled, and accomplish several goals. The base was designed with two insert aligners for holding inserts in the correct orientation relative to the fluid access holes (FIG. 5). This ensured that the contacts brought down from above would mate with the corresponding electrode mating pads in the insert. A slot was added for inserting a glass slide, providing a flat, cleanable surface on which the insert could rest. The base was designed to be detachable from the main assembly as to provide clearance for the plunger to be inserted.

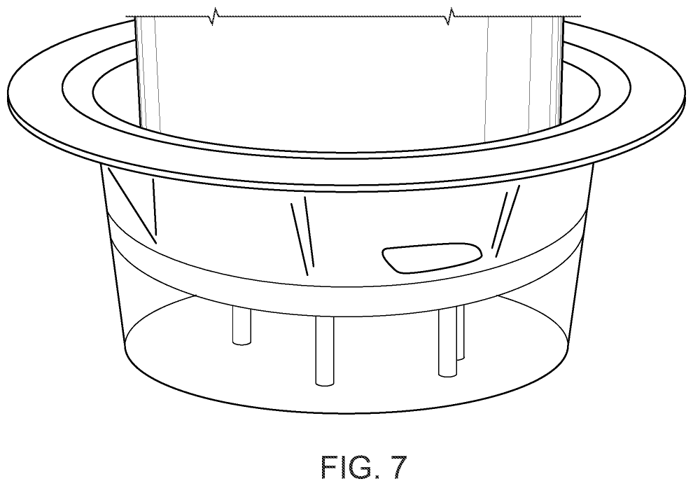

The main assembly was designed to provide stability to the whole rig, and to hold the spring-loaded plunger apparatus above the insert. The main assembly predominantly features a cylindrical housing with slots for constraining the vertical travel of the plunger, providing a way to lock it into a raised position, and a path for inserting it. The plunger, in turn, was designed to hold a circuit board containing the gold-plated contacts that make contact with the insert electrodes, using force provided by a conical compression spring (McMaster-Carr) confined between it and the main assembly, in order to form a pressure connection (FIG. 7). It also included a hollow guide shaft, for allowing wiring to pass through from the attached circuit board, and to ensure that plunger could move up and down in a consistent manner.

The circuit boards for the rig were simple in design. The board on the plunger was designed to have gold-plated contact pins added, corresponding to the mating pads of the electrode pattern, and respective wires attached. The contacts for stimuli were attached to a single stimulus connection, so that identical stimuli would always be delivered to each of the two constructs. The board on the back of the rig was made for holding several BNC jacks, and to interface them with the wires coming up from the plunger board. The BNC jacks on this board could then be easily connected to electrophysiology equipment using standard or custom cabling.

### Rig Fabrication and Assembly

The three 3D printed components were individually printed on a LulzBot TAZ 5 printer, following the same procedure described above. The two circuit board components were milled from 0.0625 in thick copper-clad garolite stock sheeting (McMaster-Carr), following a procedure similar to that described above, except with each board prepared individually with one NC file, all operations performed with a 0.040 in diameter square-ended two-flute end mill, and no lubrication applied.

The rig was assembled starting with the circuit board for the plunger. Gold-plated military-specification electrical connector pins (Mouser Electronics) were populated into the board, and soldered into place while held against a flat surface. Stranded 22 AWG copper wires were then soldered into place, so that continuous electrical connections were made from the end of the pins to the ends of the wires.

The plunger component had the conical compression spring slid onto it, with the smaller end pressed against the upper surface of the plunger. The assembled plunger circuit board's wires were threaded through the guide shaft, and then the board was fixed in place on the bottom of the plunger with hot glue.

The wires could then be threaded through the hole for the guide shaft in the main assembly, and the plunger inserted in the entry slot and rotated into place. The back circuit board was then populated with BNC jacks (Mouser Electronics), which were soldered into place. An additional grounding wire was added to connect the common ground to the Faraday cage in which the rig would be placed. Wires were threaded through the guide on the top of the main assembly, and the remaining ends were soldered into the corresponding locations on the back circuit board for the associated BNC connector. All BNC connectors were wired such that the outer casing was ground, and the inner conductor was the signal. The back circuit board and wires were then hot glued into place on the main assembly. The base component was then added to the bottom of the main assembly, and thus the final rig completed.

### Hydrogel Construct Preparation

Previous work by Curley et. al. on ultraviolet (UV) initiated micropatterned polymerization of hydrogels forms the basis of the method used for obtaining dual-hydrogel constructs [6]. Inserts with custom MEAs were removed from the incubator, and wash solutions were aspirated. The walls of the inserts were wiped with a cotton swab around the bottom with sterile filtered Rain-X, as to prevent meniscus formation. Inserts were then filled with 500 μL of sterile filtered PEG solution, prepared with the same ingredient proportions as described above. Inserts with PEG solution were then aligned with low-intensity white light on the DMD platform with the growth-restrictive pattern loaded, so that the pattern was oriented relative to the electrode pattern, as depicted by the overlaid depiction in FIG. 2. Each of the two growth restrictive gels were crosslinked, one after the other, with a 40 s application of UV light.

Excess PEG solution was then aspirated, and under a stereomicroscope, the remaining PEG solution in the voids of the growth-restricting pattern was removed with a KimWipe. The voids were then filled with approximately 10 μL of 4% HP solution, prepared as the 8% HP solution described above, but with only 0.02 g of Me-HP. The HP filled voids were then crosslinked with a 60 s application of UV light with the DMD, with a corresponding photomask loaded.

The now finished inserts with constructs were then washed 3 times with wash solution prepared as described above, 2 mL in the well below, and 1 mL above. The plate of inserts with constructs was then returned to the incubator with 2 mL of wash solution left in the well below each insert.

### Example 2: Electrophysiology with Custom Platform

### Dorsal Root Ganglion (DRG) Tissue Culture for Myelination

The DRG tissue culture method used was adapted by our lab from the Peles lab protocol to induce myelination in our DRG constructs [28]. Prior to tissue implantation, MEA inserts with constructs had wash solutions aspirated and replaced with 2 mL of neuralbasal media solution (NB), prepared with 48 mL neurobasal media, 1 mL of B-27® supplement (Gibco), 0.5 mL of GlutaMAX® (Gibco), 10 μL of 100 μg/mL nerve growth factor (NGF), and 0.5 mL of anti-anti. Inserts were left in the incubator for at least 3 hours to allow for the NB media to diffuse into the constructs.

DRG explant tissues were obtained from EIS Long Evans rat microdissection, as described previously [8]. All animal handling and tissue harvesting procedures were performed in observation of the corresponding guidelines set forth by the NIH (NIH Publication #85-23 Rev. 1985). DRGs were pushed into the round ends of the growth-permissive, HP-filled regions of each construct, with one DRG per construct.

After one day of DRG growth on NB media, the growth media was changed out for 2 mL of premyelination media (PM), consisting of 48.5 mL of Basal Eagle's Medium, 0.5 mL of ITS supplement (Gibco), 0.5 mL of GlutaMAX, 0.1 g of bovine serum albumin (BSA), 0.2 g of D-glucose, 10 μL of 100 μg/mL NGF, and 0.5 mL of anti-anti. The PM media was changed three additional times, for a total of 4 media changes over growth days 2 through 9.

For growth day 10, the media was changed for 2 mL of myelination media. This media was prepared with 41 mL of Basal Eagle's Medium, 0.5 mL of ITS supplement, 0.5 mL of GlutaMAX, 7.5 mL of fetal bovine serum (FBS), 0.2 g of D-glucose, 10 μL of 100 Kg/mL NGF, 0.5 mL of anti-anti, and 2 μg of L-ascorbic acid. This media was continued for a total of two weeks, for a total of 6 applications.

### Preparation for Electrophysiology Experiment

Active perfusion was not included in the design of the custom electrophysiology rig, and so ACSF was prepared and applied as closely as possible to that in an active perfusion setup, such as that in previous field potential recordings. ACSF solutions were prepared from a 10× stock solution, consisting of 1 L of deionized water, 72.5 g of NaCl, 3.73 g of KCl, 21.84 g of NaHCO₃, and 1.72 g of NaH₂PO₄. This stock solution was used to make 1× ACSF, where 5 mL of 10× ACSF was used, diluted with 45 mL of deionized water, and had 200 μL of 1M MgSO₄ and 100 μl of 1M CaCl₂ added. This solution was then bubbled with 95% O₂ 5% CO₂ for approximately one hour, and then the container promptly sealed at the conclusion of bubbling.

Approximately a quarter of this solution was then separated into individual 1 mL aliquots, and all ACSF solutions were left in a 37° C. heat bath until warm. 1 mM stock TTX solution was thawed out for addition into ACSF aliquots immediately prior to use.

The custom electrophysiology rig was fixed to the inner table of a Faraday cage, and three custom BNC cables were attached—two to go to two recording channels on a ML138 Octal Bio Amp attached via I²C to a PowerLab™ 8/30 (ADInstruments), corresponding to the recording electrodes on each of the two constructs per insert, and the third connected to a STG4004 stimulus generator (Multi Channel Systems MCS GmbH). The grounding wire on the back circuit board of the rig was screwed into the Faraday cage table as to ensure complete grounding. The PowerLab and the stimulus generator were wired together so that the PowerLab could trigger stimuli directly. A glass slide was washed with 70% ethanol and placed in the corresponding slot in the bottom of the rig, providing the flat surface on top of which the MEA insert would be supported.

Electrophysiology Procedure with Custom Platform

After 3 weeks, 2 days of growth, electrophysiology was conducted on the neurite constructs cultured on the custom MEA inserts Immediately prior to the electrophysiology experiment, images were obtained of both constructs in each insert to identify the amount and health of neurite growth, and also to ensure that the constructs either did or did not contain tissue.

The electrophysiology procedure was centered on the application of ACSF washes. Each insert was individually removed from the incubator, had its growth media removed, was moved to a new transportation plate, and was subjected to a series of ACSF washes. For washes of normal ACSF, 1 mL of ACSF was gently pipetted on the top of the insert, in direct contact with the constructs, and then the insert, inside the transportation plate, was placed into a rotating incubator, rotating at 60 rpm, and at a temperature of 37° C. ACSF solution was removed from the heat bath immediately prior to use, and sealed and returned immediately after use. For ACSF with added TTX (ACSF-TTX), an individual ACSF aliquot was removed from the heat bath, and had either 0.5 μL or 1 μL of 1 mM TTX concentrate added immediately prior to use, corresponding to 0.5 μM and 1 μM TTX concentrations, respectively. After a given amount of time incubating with ACSF, the plate was removed from the incubator, the ACSF aspirated with the pipette, and the insert placed into the electrophysiology rig, with the fluid access holes slid onto the two aligners, and the ground electrode contact pointing away from the rig. The rig plunger was then twisted out of its locked position, and gently lowered to make contact with the electrode mating contacts.

The recording software used was LabChart (ADInstruments), which interfaced with the PowerLab. The stimulus generator was programmed using its control software, MC_Stimulus II (Multi Channel Systems MCS GmbH). LabChart was programmed to, when manually triggered, provide a triggering signal to the stimulus generator, which in turn was programmed with MC_Stimulus II to output a −50 mV, 200 μs pulse stimulus when triggered, then return to ground potential. LabChart was set to record in the 1 mV range for 200 ms for each manual triggering.

After placing an insert into the rig, the combined stimulus and recording was manually triggered at least 50 times, sometimes less when responses visibly fatigued prior to reaching 50. The delays between triggering varied between 0.5-5 s, but were mostly on the short end of that range.

Table 2 below shows the final ACSF wash procedure used, and descriptions of the purposes of each wash. The wash procedure was designed to allow for ample wash-in and wash-out time for ACSF and TTX, to aid in confirming biological origin of any responses.

TABLE 2
Electrophysiology wash procedure

| Wash number | Type | Duration | Description |
|---|---|---|---|
| 1 | ACSF | 5 min | Attaining response |
| 2 | ACSF | 2 min | |
| 3 | ACSF | 2 min | |
| 4 | ACSF | 5 min | Baseline response |
| 5 | ACSF-TTX, 0.5 μM | 5 min | TTX wash-in |
| 6 | ACSF-TTX, 1 μM | 5 min | TTX response |
| 7 | ACSF | 5 min | TTX wash-out |
| 8 | ACSF | 5 min | Post-TTX response |

Data obtained after each of washes 4, 6, and 8 were used for verifying the biological origin of responses.

Responses were counted for the electrophysiology data produced after each wash, where the count was the number of responses seen within 50 stimuli of the first visible response. Responses were confirmed to be biological in origin when responses were seen after washes 4 and 8, the baseline and post-TTX responses, but not after wash 6, corresponding to saturation of TTX.

### Impedance Analysis of Hybrid Conductive Hydrogels

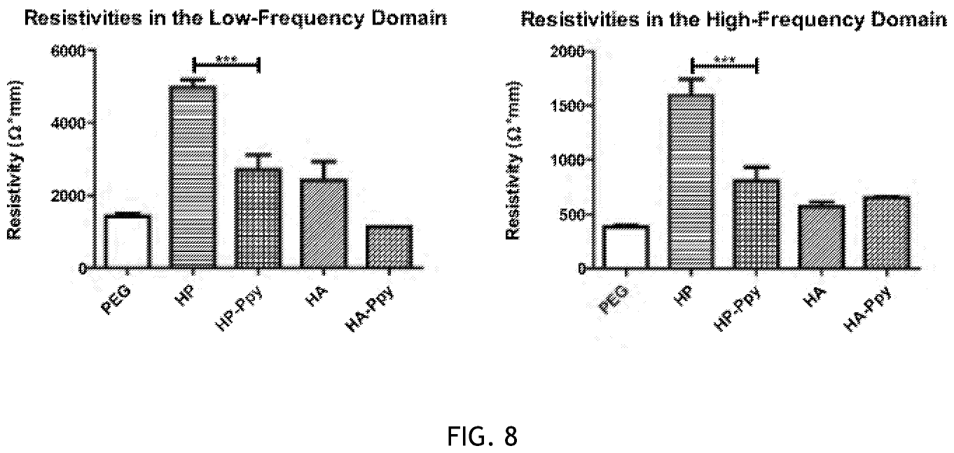

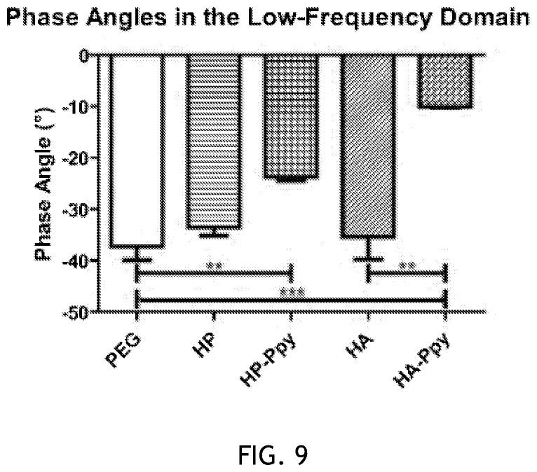

For the hydrogels analyzed, resistivities were obtained and analyzed in the low-frequency domain, 100 Hz, and the high-frequency domain, 1 MHz (FIG. 8). Phase angles were obtained and analyzed in the low-frequency domain (FIG. 9). For PEG, n=10; for HP, n=4; for HP-Ppy, n=6; for HA, n=4; and for HA-Ppy, n=2. One-way ANOVA showed significance in each of the low and high-frequency domains of resistivity (p<0.0001 for both), as well as in the low-frequency domain of phase angle (p=0.0001). Bonferroni post-hoc tests showed Ppy addition did reliably reduce resistivity of HP in the low and high-frequency domains (p<0.001 for both), but did not for HA, and no gel achieved a lower resistivity than that of PEG.

Bonferroni post-hoc tests showed that Ppy addition reliably reduced (brought closer to zero) phase angles of HA (p<0.01), but not those of HP. The phase angles of HP-Ppy and HA-Ppy were both reduced from that of PEG (p<0.01, and p<0.001, respectively).

Electrophysiology with Custom Platform

4 sets of 6 inserts were prepared with custom MEAs, constructs, and DRG tissues. Of these, 3 of the sets were prepared using the final e-beam technique. The first set of inserts did not have measures taken to mitigate leftover metal particulates, those being taping exposed areas of the inserts, and soaking in wash solution post-deposition. This set visibly was of poor health after one week of culture, and so was halted.

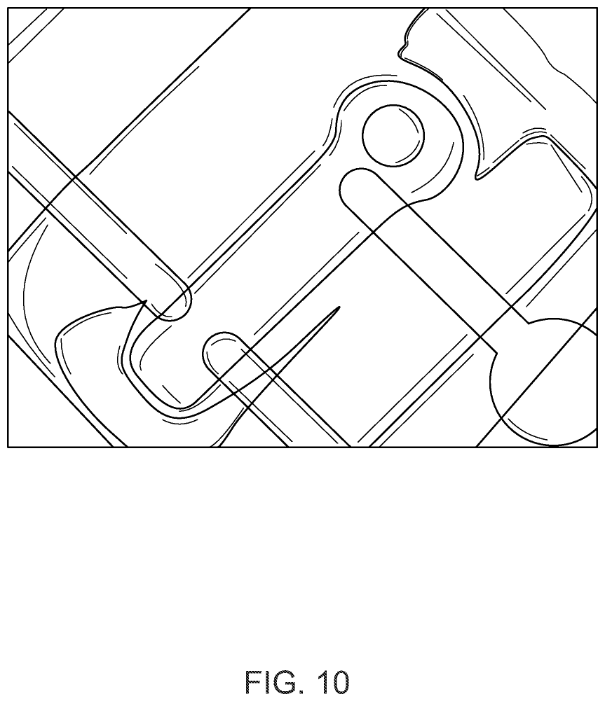

Out of the remaining 3 sets of inserts with viable tissues, a total of 7 inserts, or 14 constructs, were subjected to the finalized ACSF wash procedure. 2 of these constructs were empty, and at least one response was seen in the trials counted for biological verification of 9 of the remaining 12 constructs. Of these, 3 constructs' responses were confirmed to be biological in origin with TTX (FIG. 10). The notation used for identifying individual constructs was S(set number)-(insert number)-(construct number) (See Table 3 below).

TABLE 3
Confirmed biological responses

| Construct | Baseline responses | TTX responses | Post-TTX responses |
|---|---|---|---|
| S2-3-1 | 4 | 0 | 3 |
| S3-4-2 | 1 | 0 | 3 |
| S4-5-2 | 2 | 0 | 2 |

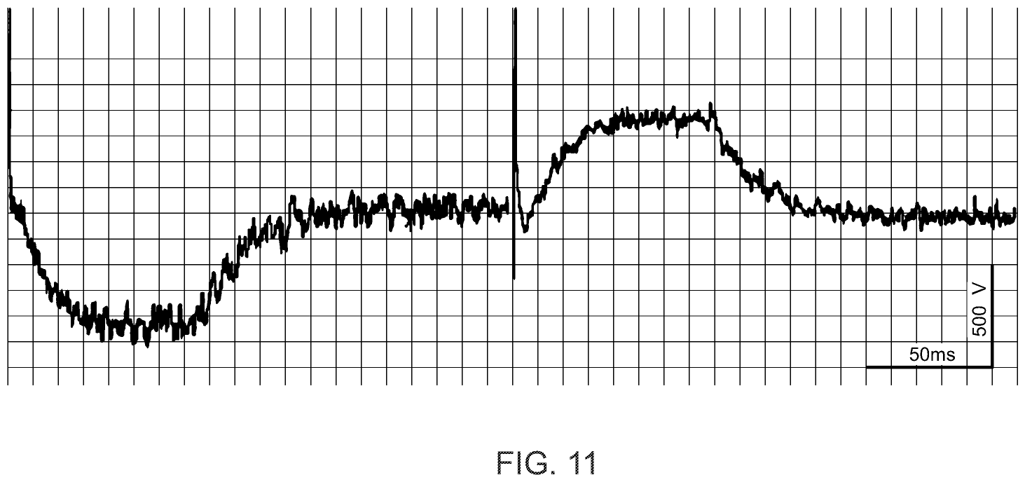

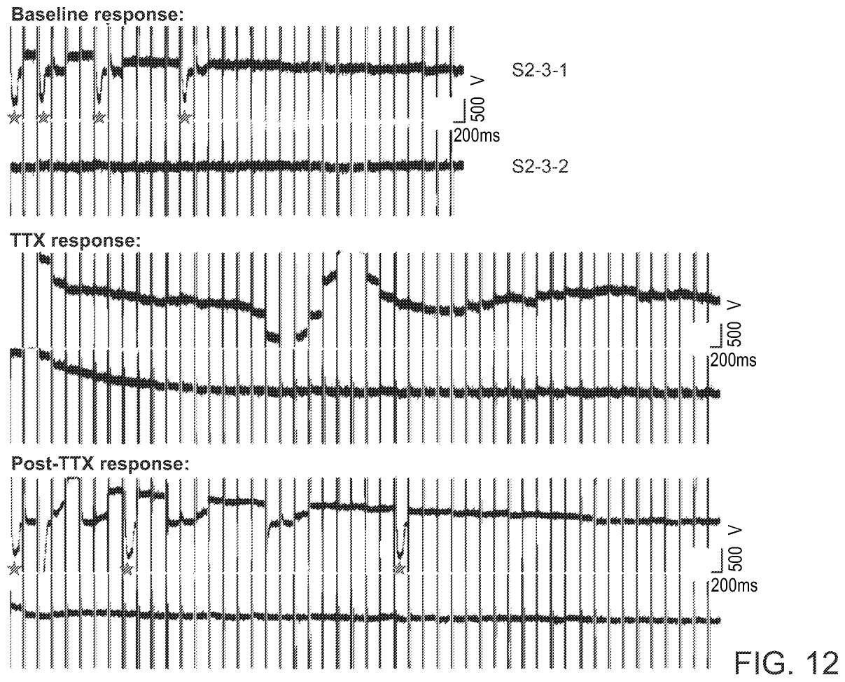

Of these confirmed biological responses, two expressed negative responses, those being S2-3-1 and S3-4-2, and one expressed positive responses, that being S4-5-2. The curve characteristics of both the positive and negative responses were similar, and appeared to be inverses of one another (FIG. 11). Responses, both positive and negative, had magnitudes of approximately 500 μV, and were approximately 100 ms in duration (FIG. 12).

The confirmed biologically responding construct S2-3-1 was contained in an insert with an empty construct, S2-3-2. This served as a negative control, and no responses were seen in S2-3-2.

### Discussion

EIS results showed that no hydrogel, or hybrid conductive hydrogel, had lower resistivities than PEG. A resistivity an order of magnitude lower than that of PEG was considered a suitable result for a hybrid gel, yet no gel managed to surpass PEG's conductivity.

Despite lacking the functionality desired, addition of the conductive Ppy polymer to HP hydrogels was shown to reliably cause a drop in resistivities in the high and low frequency domains. It was not shown to do the same in HA, however, the phase angles of both HA and HP were reduced (made closer to zero) to substantially less than those of PEG by the addition of Ppy. This result shows that HP-Ppy may be somewhat more advantageous to conducting electricity than HP. However, for conducting a signal, the phase angle is likely a more important criterion, as lower phase angles (closer to zero) correspond with lower signal distortion. This means that HA-Ppy would likely conduct a given signal with less distortion that PEG or HP-Ppy, as it had the phase angle closest to zero. CAPs seen in prior electrophysiological experiments have had a characteristic 10 ms signal[13], which corresponds to a frequency of 100 Hz, within the low-frequency domain. HA-Ppy would thus likely perform better than PEG in CAP conduction, as it had a similar resistivity PEG in the low-frequency domain, but a lower phase angle, meaning that it would be just as suited as a path for current to flow, and would do so with less disruption of the characteristics of the signal.

A possible explanation for this result is that the preparation of HA-Ppy resulted in the formation interpenetrating polymer network (IPN), within which the HA and Ppy would not be chemically associated or bound, allowing for Ppy to conduct a signal unhindered. It may be possible that this did not occur fully within the HP-Ppy, resulting in a phase characteristic similar to that of HP alone. Candidate chemical associations between the HP and Ppy that would prevent IPN formation include hydrogen bonding, acid/base interaction, peptide bonding, and the crosslinking between HP's methacrylate groups and pyrrole. Many of the possible chemical associations between HP and Ppy can be summarized as being related to HP's high negative charge density, and Ppy's relative positive charge. HP's higher charge density than HA would implicate more of these interactions between HP and Ppy, thereby hindering charge and signal propagation.

Overall, hybrid conductive hydrogels were determined to an ineffective solution to facilitate the electrical connection between the neurites in hydrogel constructs and electrodes. While there is some possibility that signals conducted in these hybrid gels would be less distorted, the overall effort required to emplace the added conducting polymer was too great to justify its minimal resistivity improvements in this application.

Adding the hybrid gels into selected regions of neurite constructs was found to require a multistep process that significantly increased the time required to produce the constructs using the DMD. Furthermore, preliminary experiments with added DRG tissues appeared to show that the hybrid gels were not conducive to neurite growth.

These limitations found in hybrid conductive hydrogels as applied to neurite electrophysiology were confirmed by observations found by collaborators[51]. The relatively small advantages of hybrid conductive hydrogels, when compared to the efforts required to produce them, led to their abandonment as an addition to hydrogel neurite constructs for facilitating electrophysiological evaluation. Despite this, these hybrid gels show potential for use in other applications in which a compliant, hydrated conducting material is required.

### Rapid Electrophysiology Platform

The rapid electrophysiology platform developed in this work was shown to be able to readily record biological signals from hydrogel neurite constructs. Of the constructs tested using the finalized wash procedure, 3 were confirmed to provide responses of biological origin, due to muting of the responses with TTX. The biological responses seen were much longer than those seen previously, where previous work by our lab has shown CAP responses to be 5-10 ms [13], the responses seen in this work were approximately 100 ms. This was to be expected, as previous field-potential recording electrodes were a fine point, whereas the recording electrodes in this work were 500 m. This would result in recordings becoming the sum of a greater length of more axons, which means that more, differently-timed axonal responses would be included in the recorded signal.

One unexpected result seen during electrophysiological testing was the presence of both positive and negative biological responses, as opposed the anticipated negative only responses. Positive CAPs have been shown in literature [30], [31], and two possible explanations exist for this phenomenon. The simplest is that positive responses seen were intercellular measurements. This however seems unlikely, as the MEAs had no means of entering the axonal lumen. Another more likely explanation is that the neurite fiber direction and orientation was not as expected, resulting in the distal end of the stimulated fiber not being ground-referenced correctly, or as anticipated. Differing placement of the grounding electrode relative to the neurite tissue would result in a different “zero” point, and so could result in inverted voltages, similar to how a battery can be measured to have a positive or negative voltage depending on which voltmeter probe is placed where. This seems to be the most likely scenario, as the response magnitudes seen, 5000 μV, correspond with those seen previously with field potential recording electrodes[13]. Also, the construct with the confirmed biological positive response, S4-5-2, can be seen to have large neurite bundles growing outside of the PEG channel in figure 4.4, which would not be properly ground-referenced. A future change that could possibly prevent positive responses from appearing would be a PEG gel with a more constrained channel, preventing more stray neurite propagation to an improperly grounded location.

With the custom MEA and interfacing rig, electrode connection went from a minutes-long procedure with field potential recording electrodes to a roughly five-second one. This shows that the custom MEA platform shows high capability of being automated in the future, and so accomplished a major portion of its goal. The most apparent drawback of the platform design as presented in this work is the lack of an active perfusion system. A perfusion system would provide a constant supply of fresh ions and other factors to the constructs on the cell culture insert, by pumping in fresh ACSF while removing used ACSF. Such a pump setup would also allow for easy addition of factors that would change the electrophysiological response, such as TTX as used here, or pharmaceutical agents in the future. A perfusion system was included in previous field-potential recording experiments by our lab[13], but was not included in the platform developed in this work, in order to reduce the complexity of the required rig. Without a continuous supply of fresh ACSF, and instead relying on the manual wash technique used in this work, resulted in electrophysiological responses disappearing relatively quickly due to fatigue. Wash-in and wash-out of TTX also proved to be difficult, requiring multiple wash steps in order to be effective, which in turn required a substantial increase of manual effort. By adding an active perfusion system, electrophysiology with the platform designed in this work would be a much more practical and automatable process.

One issue that could arise from the addition of an active perfusion system is current shunting, where ACSF essentially shorts-out the surface electrodes. This can be the source of problems in some electrophysiology experiments, and some solutions have been developed that do not apply well to this work [32]. This shunting effect was avoided by removing the excess ACSF solution from inserts after each wash, prior to placing them in the rig. However, with an active perfusion system, a level of ACSF would be maintained inside the insert. This could be mitigated by having the pump adding ACSF temporarily turned off while the pump removing it is left on, allowing the excess to be removed, and providing a gap of time in which to run the electrophysiology trial.

Another solution to the current shunting problem would be to insulate the electrodes, and move the point of contact with the interfacing rig away from the area in which the ACSF solution is present. There are normal features of commercial MEAs [34]. This could be accomplished in many different ways, but would likely require replacement of the cell culture inserts used in this work as the flat bottom membrane is not practical for having both neurite constructs surrounded with ACSF, and contacts being kept dry.

### REFERENCES

1. World Health Organization, Neurological disorders: public health challenges. Geneva,

Switzerland: WHO Press, 2006.
2. S. B. Rutkove. A 52-year-old woman with disabling peripheral neuropathy: review of diabetic polyneuropathy. JAMA, vol. 302, no. 13, pp. 1451-1458, 2009.
3. Centers for Disease Control and Prevention. “Number (in Thousands) of Hospital Discharges with Peripheral Arterial Disease (PAD), Ulcer/Inflammation/Infection (ULCER), or Neuropathy as First—Listed Diagnosis and Diabetes as Any—Listed Diagnosis United States, 1988-2007.” Internet: http://www.cdc.gov/diabetes/statistics/hosplea/diabetes_complications/fig1_number.htm, 2014 [Apr. 8, 2016].
4. A. H. Boecker, et al. Pre-differentiation of mesenchymal stromal cells in combination with a microstructured nerve guide supports peripheral nerve regeneration in the rat sciatic nerve model. Eur. I Neurosci., vol. 43, pp. 404-416, 2015.
5. M. Shahraki, et al. Influence of Tacrolimus (FK506) on Nerve Regeneration Using Allografts: A Rat Sciatic Nerve Model. J. Oral Maxillofac. Surg., 73(7): 1438.e1-1438.e9, 2015.
6. J. L. Curley and M. J. Moore. Facile micropatterning of dual hydrogel systems for 3D models of neurite outgrowth. J. Biomed. Mater. Res.—Part A, vol. 99 A, no. 4, pp. 532-543, 2011.
7. J. Lee, M. J. Cuddihy, and N. a Kotov. Three-dimensional cell culture matrices: state of the art. Tissue Eng. Part B. Rev., vol. 14, no. 1, pp. 61-86, 2008.
8. J. L. Curley, S. R. Jennings, and M. J. Moore. Fabrication of Micropatterned Hydrogels for Neural Culture Systems using Dynamic Mask Projection Photolithography.,” J. Vis. Exp., no. 48, pp. 1-6, 2011.
9. Z. Wang, et al. Enzyme-induced vitreolysis can alleviate the progression of diabetic retinopathy through the HIF-la pathway,” Investig. Ophthalmol. Vis. Sci., vol. 54, no. 7, pp. 4964-4970, 2013.
10. O. J. Vilholm, A. A. Christensen, A. H. Zedan, and M. Itani. Drug-induced peripheral neuropathy,” Basic Clin. Pharmacol. Toxicol., vol. 115, no. 2, pp. 185-192, 2014.
11. J. H. Leal-Cardoso, et al. Linalool blocks excitability in peripheral nerves and voltage-dependent Na+ current in dissociated dorsal root ganglia neurons,” Eur. I Pharmacol., vol. 645, no. 1-3, pp. 86-93, 2010.
12. V. Khori, A. Alizadeh, and M. Niknam. Dynamic age-related changes of extracellular field potential of isolated AV-node of rabbit. Physiol. and Pharmacol, vol. 15, no. 2, pp. 173-181, 2011.
13. R. Huval. 3D microengineered hydrogels as a novel assay for electrophysiological investigation of biomimetic neural cultures. M.S. thesis, Tulane University, LA, 2013.
14. M. Scanziani and M. Hausser. Electrophysiology in the age of light. Nature, vol. 461, no. 7266, pp. 930-939, 2009.
15. K. S. M. Smalley, M. Lioni, and M. Herlyn. Life isn't flat: taking cancer biology to the next dimension. In Vitro Cell. Dev. Biol. Anim., vol. 42, no. 8-9, pp. 242-247, 2006.
16. J. A. Pedersen and M. A. Swartz. Mechanobiology in the third dimension. Ann. Biomed. Eng., vol. 33, no. 11, pp. 1469-1490, 2005.
17. M. C. Cushing and K. S. Anseth. Materials science. Hydrogel cell cultures. Science, vol. 316, no. 5828, pp. 1133-4, 2007.
18. R. P. Seisyan. Nanolithography in microelectronics: A review. Tech. Phys., vol. 56, no. 8, pp. 1061-1073, 2011.
19. T. Billiet, et al. A review of trends and limitations in hydrogel-rapid prototyping for tissue engineering. Biomaterials, vol. 33, no. 26, pp. 6020-41, 2012.
20. J. A. Majdi. New digital light projection microscopy techniques for studying optogenetic activation in neurons. M.S. thesis, Tulane University, LA, 2012.
21. P. M. Conn, Neuroscience in medicine: Third edition. Totowa, N.J.: Humana Press, 2008.
22. A. Scuteri, A. Cassetti, and G. Tredici. Adult mesenchymal stem cells rescue dorsal root ganglia neurons from dying,” Brain Res., vol. 1116, no. 1, pp. 75-81, 2006.
23. K. Takahashi, M. Ishida, and H. Takahashi. Expression of Sema3D in subsets of neurons in the developing dorsal root ganglia of the rat. Neurosci. Lett., vol. 455, no. 1, pp. 17-21, 2009.
24. R. M. Huval, et al. Microengineered Peripheral Nerve-on-a-Chip for Preclinical Physiological Testing,” Lab Chip, vol. 15, no. 10, pp. 2221-2232, 2015.
25. M. Pruginin-Bluger, D. L. Shelton, and C. Kalcheim. A paracrine effect for neuron-derived BDNF in development of dorsal root ganglia: stimulation of Schwann cell myelin protein expression by glial cells.,” Mech. Dev., vol. 61, no. 1-2, pp. 99-111, 1997.
26. L. Nobbio, et al. PMP22 transgenic dorsal root ganglia cultures show myelin abnormalities similar to those of human CMT1A. Ann. Neurol., 50(1), pp. 47-55, 2001.
27. M. Stettner, et al. A reliable in vitro model for studying peripheral nerve myelination in mouse. J. Neurosci. Methods, vol. 214, no. 1, pp. 69-79, 2013.
28. Y. Eshed, et al. Gliomedin mediates Schwann cell-axon interaction and the molecular assembly of the nodes of Ranvier. Neuron, vol. 47, no. 2, pp. 215-229, 2005.
29. The McGill Physiology Virtual Lab. Characteristics of the CAP. Internet: http://www.medicine.mcgill.ca/physio/vlab/cap/character.htm, 2005 [Apr. 8, 2016].
30. W. Lai and N. Dillier. A simple two-component model of the electrically evoked compound action potential in the human cochlea. Audiol. Neurotol., pp. 333-345, 2000.
31. B. R. Noga, P. a Fortier, D. J. Kriellaars, X. Dai, G. R. Detillieux, and L. M. Jordan. Field potential mapping of neurons in the lumbar spinal cord activated following stimulation of the mesencephalic locomotor region. I Neurosci., vol. 15, no. 3 Pt 2, pp. 2203-2217,1995.
32. A. A. Velumian, et al. Modular double sucrose gap apparatus for improved recording of compound action potentials from rat and mouse spinal cord white matter preparations. J. Neurosci. Methods, vol. 187, no. 1, pp. 33-40, 2010.
33. H. Tankisi, et al. Correlation between compound muscle action potential amplitude and duration in axonal and demyelinating polyneuropathy. Clin. Neurophysiol., vol. 123, no. 10, pp. 2099-2105, 2012.
34. M. G. Liu, et al. Use of multi-electrode array recordings in studies of network synaptic plasticity in both time and space. Neurosci. Bull., vol. 28, no. 4, pp. 409-422, 2012.
35. M. Suzuki, et al. Neuronal cell patterning on a multielectrode array for a network analysis platform,” Biomaterials, vol. 34, no. 21, pp. 5210-5217, 2013.
36. Multi Channel Systems MCS GmbH. “USB MEA Systems.” Internet: http://www.multichannelsystems.com/products/usb-mea-systems, [Apr. 8, 2016].
37. K. Kanazawa, A. Diaz, M. Krounbi, and G. Street. Electrical properties of pyrrole and its copolymers,” Synth. Met., vol. 4, pp. 119-130, 1981.
38. B. C. Kim. The synthesis and characterisation of hydrogel and polypyrrole blends. PhD dissertation, University of Wollongong, Wollongong, New South Wales, Australia, 1999.
39. S. Brahim, et al. Amperometric determination of cholesterol in serum using a biosensor of cholesterol oxidase contained within a polypyrrole-hydrogel membrane. Anal. Chim. Acta, vol. 448, no. 1-2, pp. 27-36, 2001.
40. Novocontrol Technologies. “Impedance Measurement,” Internet: http://novocontrol.de/html/introjmp_spectr.htm, [Apr. 8, 2016].
41. Corning Incorporated. “Transwell ® Permeable Supports Selection and Use Guide.” Internet: http://csmedia2.corning.com/LifeSciences/Media/pdf/transwell_guide.pdf, 2013 [Apr. 8, 2016].
42. Attend Technology Incorporated. “USB 2.0 Connector with USB Type A Female, DIP for Right Angle Type.” Internet: http://attend.manufacturer.globalsources.com/si/6008814768084/pdt1/USB2.0/1052715231/USB-2.0-Connector.htm, [Apr. 8, 2016].
43. M. Braddock. “Electric DC motors (type, function and application to robots).” Internet: http://www2.mae.ufl.edu/designlab/Class%20Projects/Background%20Information/Electric%20DC%20motors.htm, [Apr. 8, 2016].
44. D. M. Mattox. Handbook of Physical Vapor Deposition (PVD) Processing. 2nd ed. Kidlington, Oxford, UK: Elsevier Incorporated, 2010.
45. S. Mohtashami. Electrochemical Properties of Flexible Electrodes for Implanted Neuromuscular Excitation Applications. M.S. thesis, McMaster University, Hamilton, ON, 2011.
46. T. Rowe Price. “A brief history of 3D printing.” Internet: http://individual.troweprice.comlstaticFiles/Retail/Shared/PDFs/3D PrintingInfographic_FINAL.pdf, 2011 [Apr. 8, 2016].
47. 3D Printing for Beginners. “3D Printers—How Do They Work?,” Internet: http://3dprintingforbeginners.com/wp-content/uploads/2014/04/3D-Printing-Technology_Download.pdf, [Apr. 8, 2016].
48. MISUMI Corporation. “Glass Transition Temperature Tg of Plastics.” Internet: http://www.misumi-techcentral.com/Wen/mold/2011/12/106-glass-transitiontemperature-tg-of-plastics.html, 2011 [Apr. 8, 2016].
49. Thomas Publishing Company. “More about CNC Milling.” Internet: http://www.thomasnet.com/about/cnc-milling-51276103.html, [Apr. 8, 2016].
50. P. Khoshakhlagh. A hydrogel tool-kit for in vitro neural regeneration models. PhD dissertation, Tulane University, New Orleans, La., 2015.
51. C. Bettinger. Personal communication concerning unpublished observations. Carnegie Mellon University, Pittsburgh, Pa.

1. An insert comprising:
(i) a permeable solid support comprising a top surface and a bottom surface; the top surface horizontal or substantially horizontal relative to a surface onto which the bottom surface of the insert lies, the top surface divided into an inner portion and an outer portion by one or a plurality of protrusions extending vertically from the top surface; wherein at least one region of the inner portion of the top surface defines the bottom face of a vessel and wherein the one or plurality of protrusions define one or more contiguous sidewalls of the vessel;
(ii) one or plurality of electrodes physically attached to the top surface of the permeable solid support and positioned within the vessel; and
(iii) one or plurality of contact pads, positioned on top of the at least one region of the outer portion of the top surface.

2. The insert of claim 1, wherein:
(i) the one or plurality of electrodes are planar in shape with a top and a bottom surface, the bottom surface of the one or plurality of electrodes positioned adjacent or substantially adjacent to the bottom face of the vessel;
(ii) the one or plurality of electrodes comprise one or more of titanium, gold, stainless steel, platinum, iridium, tungsten, carbon fiber, silver, or silver chloride;
(iii) the one or plurality of electrodes are microelectrodes;
(iv) the flexular modulus of the permeable solid support is from about 0.2 to about 20 Gigapascals (GP);
(v) the permeable solid support comprises a plurality of pores from about 0.1 μm to about 3 μm in diameter; and/or
(vi) the permeable solid support comprises polyester or polyvinyl polymers.

3. (canceled)

4. The insert of claim 1, wherein:
(i) the insert comprises a first electrode and a second electrode, the first and second electrodes aligned in parallel in respect to a longitudinal axis but positioned proximate to opposite facing surface of the sidewalls; and/or
(ii) the insert comprises a first protrusion that is circular or substantially circular physically attached to the top surface on its edge defining the sidewalls of the vessel with a height from about 1 millimeter to about 10 millimeters above the top surface, wherein, the insert further comprises:
(a) a circular or semi-circular ring affixed to the permeable solid support, such that the permeable solid support and the ring define a cylindrical or substantially cylindrical vessel with a height from about 0.5 to about 15 millimeters; and/or
(b) a hydrogel matrix layer positioned across the bottom face of the vessel and wherein at least one portion of the electrode is positioned below a top surface of the hydrogel matrix layer or protruding just above a top layer of the hydrogel matrix layer.

5-8. (canceled)

9. The insert of claim 4, wherein the hydrogel matrix forms a layer with a height from about 5 to about 500 microns; and wherein the hydrogel matrix comprises a cavity with a depth from about 5 to about 500 microns and wherein the bottom region of the cavity has a surface area of from about 500 to about 5000 square microns.

10. The insert of claim 1, further comprising:
one or a plurality of isolated Schwann cells; and
one or a plurality of dorsal root ganglion (DRG) or DRG fragments.

11. The insert of claim 10, wherein a first hydrogel matrix is layered across the top surface and comprises at least a first cavity, the cavity comprising a contiguous side region and a bottom region;
wherein at least one portion of the electrode is positioned below the bottom region or protruding minimally above the bottom region;
wherein the one or plurality of isolated Schwann cells and/or the one or plurality of DRG or DRG fragments is positioned on top of the bottom region of the cavity such that the Schwann cells, DRG or DRG fragments are positioned above or are in contact with the one or plurality of electrodes.

12-13. (canceled)

14. The insert of claim 4, wherein:
(i) the hydrogel matrix comprises a hydrogel of a first polymer that comprises a stiffness sufficient to prevent growth and/or cell migration and a hydrogel of a second polymer that comprises a stiffness sufficient to allow axon growth and/or cell migration;
(ii) the hydrogel matrix comprises a first polymer comprising no greater than about 15% PEG and from about 0.05% to about 5.0% of one or a combination of self-assembling peptides chosen from: RAD 16-I, RAD 16-II, EAK 16-I, EAK 16-II, and dEAK 16, and gelatin methacrylate;
(iii) the hydrogel matrix comprises one or a combination of compounds chosen from: polyethylene glycol (PEG), Puramatrix, methacrylated hyaluronic acid, agarose, methacrylated heparin, pyrrole (Py), oxidized polypyrrole (Ppy), and methacrylated dextran;
(iv) the hydrogel matrix comprises polyethylene glycol (PEG) at a concentration of no more than about 20% weight to volume (w/v) of the solution; and/or
(v) the hydrogel matrix comprises at least one cell-penetrable polymer at a concentration of from about 0.1% to about 3.0% in weight to volume (w/v) of the solution.

15-20. (canceled)

21. The insert of claim 1, wherein:
(i) the one or plurality of electrodes are in a substantially horizontal orientation on a top surface of the permeable solid support; and/or
(ii) the one or plurality of electrodes comprise at least one stimulating electrode, at least one recording electrode, and at least one ground electrode, optionally:
(a) the at least one stimulating electrode and the at least one recording electrode are at a distance from about 1 μm to about 1 cm apart;
(b) the at least one stimulating electrode and the at least one recording electrode are orientated substantially parallel to and spaced from each other; and/or
(c) the at least one ground electrode comprises a first portion oriented substantially parallel with and spaced from the at least one stimulating electrode, and the at least one ground electrode comprises a second portion oriented substantially perpendicularly relative to the at least one stimulating electrode.

22-30. (canceled)

31. The insert of claim 1 further comprising one or a plurality of cells and culture medium, wherein the one or a plurality of cells comprise one or a combination of cells and/or tissues chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, an astrocyte, an oligodendrocyte, a Schwann cell, a microglial cell, an ependymal cell, a radial glial cell, a satellite cell, an enteric glial cell, a pituicyte, an immune cell, a dorsal root ganglia, and combinations thereof.

32-33. (canceled)

34. An adapter comprising:
(i) a body defining a substantially flat and planar configuration with a top surface and a bottom surface;
(ii) one or plurality of planar electrodes on the top surface of the body;
(iii) a layer of insulating material; and
(iv) a circular or substantially cylindrical collar positioned on its edge around a central opening formed and extending through the body,
wherein the adapter comprises a pattern of contact pins radially disposed around the central opening and extending through the body, each of the contact pins electrically connected to at least one of the planar electrodes.

35. The adapter of claim 34, wherein:
(i) the body comprises a polymer resin;
(ii) the body comprises a first side edge and a second side edge, each dimensioned about 49 mm;
(iii) the body comprises a height dimensioned about 1 mm;
(iv) a central opening formed and extending through the body;
(v) the one or plurality of planar electrodes are disposed on the top surface of the body in a substantially square pattern spaced from a perimeter of the body;
(vi) the pattern of the one or plurality of planar electrodes surrounds the central opening;
(vii) the one or plurality of planar electrodes are configured to be attached to contacts of a plunger plate, the one or plurality of planar electrodes operably and electrically connected to an amplifier and current source through the contacts of the plunger plate; and/or
(viii) the one or plurality of planar electrodes form a continuous electrical connection perimeter along the top surface of the body.

36-43. (canceled)

44. A system comprising:
(i) the insert of claim 1 positioned within the central opening;
(ii) an adapter comprising:
(a) a body defining a substantially flat and planar configuration with a top surface and a bottom surface;
(b) one or plurality of planar electrodes on the top surface of the body;
(c) a layer of insulating material; and
(d) a circular or substantially cylindrical collar positioned on its edge around a central opening formed and extending through the body;
(iii) an amplifier comprising a generator for electrical current; and
(iv) a voltmeter and/or ammeter;
wherein the amplifier, voltmeter and/or ammeter, and electrodes are electrically connected to each other via a circuit.

45. The system of claim 44, further comprising one or a combination of: controller, a recording device, a computer storage memory and a screen; wherein the screen if connected to the voltmeter and/or ammeter and is capable of displaying recording measurements from the one or plurality of electrodes.

46. A system comprising:
(i) the insert of claim 1; and
(ii) a tissue culture support dimensioned to receive the insert.

47. The system of claim 46, wherein the tissue culture support comprises 1, 6, 12, 24 or 48 wells, and the insert is configured and dimensioned to be at least partially introduced into a single well.

48. (canceled)

49. A method of producing a three-dimensional culture of one or a plurality of cells in a vessel, said method comprising:
(i) contacting one or a plurality of cells with the permeable solid support of the insert of claim 1;
(ii) seeding one or a plurality of isolated cells or tissue explants comprising cells to the vessel of the insert; and
(iii) applying a cell medium into the vessel with a volume of cell medium sufficient to cover the cells.

50. A method of testing of one or a plurality of cells, comprising:
(i) positioning the one or plurality of cells on the permeable solid support of the insert of claim 1;
(ii) applying an input current or voltage to the one or plurality of electrodes of the insert;
(iii) recording an output characteristic associated with the one or plurality of cells, optionally the output characteristic comprises at least one of resistance or output current and
(iv) optionally comparing the input current or voltage to the output characteristic.

51-52. (canceled)

53. A method of testing of one or a plurality of cells, comprising:
(i) positioning the one or plurality of cells on the top surface of the adapter of claim 34;
(ii) applying an input current to the one or plurality of planar electrodes of the adapter; and
(iii) recording an output characteristic associated with the one or plurality of cells.

54. A system, comprising:
(i) a testing rig configured to receive the insert of claim 1, the testing rig comprising a body with a housing and an inner passage extending through the housing;
(ii) a plunger movably disposed within the inner passage and configured to be positioned in a raised position spaced from the insert or a lowered position disposed against the insert.

55. The system of claim 54, wherein:
(i) the testing rig comprises a base with two aligners extending therefrom, the aligners configured to receive and maintain an orientation of the insert
(ii) the base comprises a slot extending therethrough and the testing rig comprises a slide configured to be positioned within the slot of the base;
(iii) the testing rig comprises a spring disposed between the plunger and the housing, the spring urging the plunger towards the insert;
(iv) the plunger is configured to travel along a vertical axis between the raised and lowered positions;
(v) the plunger comprises a bottom end with a plate and a rod extending perpendicularly from the bottom end; and/or
(vi) the plate of the plunger comprises a circuit board with electrical contacts configured to be placed in electrical contact with the electrodes of the insert, optionally the system further comprises at least one or combination of: a recording device, an amplifier, an electricity source, a controller, a user interface, a voltmeter, and an ammeter electrically connected to the testing rig.

56-61. (canceled)

62. The system of claim 44 further comprising:
at least one of: (i) an amplifier comprising a generator for electrical current; (ii) a voltmeter; or (iii) an ammeter;
wherein the electrodes of the insert are electrically connected to the electrodes of the adapter; and
wherein the electrodes of the adapter are operably linked to a circuit and at least one of the amplifier, the voltmeter, or the ammeter.

63. A method of assessing a response from one or more cells using the system of claim 62, wherein the method comprises:
(a) growing the one or more cells on the permeable solid support of the insert;
(b) positioning the insert into the adapter;
(c) placing the adapter in the system;
(d) introducing one or more stimuli to the one or more cells; and
(e) measuring one or more responses from the one or more cells to the one or more stimuli.

64. A method of evaluating the toxicity of an agent comprising:
(a) culturing one or more cells and/or one or more tissue explants on the permeable solid support of the insert of claim 1;
(b) exposing at least one agent to the one or more cells and/or one or more tissue explants;
(c) measuring and/or observing one or more morphometric changes of the one or more cells and/or one or more tissue explants; and
(d) correlating one or more morphometric changes of the one or more cells and/or one or more tissue explants with the toxicity of the agent, such that, if the morphometric changes are indicative of decreased cell viability, the agent is characterized as toxic and, if the morphometric changes are indicative of unchanged or increased cell viability, the agent is characterized as non-toxic.

65. A method of measuring myelination or demyelination of one or more axons of one or a plurality of neuronal cells and/or one or a plurality of tissue explants, said method comprising:
(a) culturing one or more neuronal cells and/or one or a plurality of tissue explants on the permeable solid support of the insert of claim 1 for a time and under conditions sufficient to grow at least one axon; and
(b) detecting the amount of myelination on one or a plurality of axons of the one or more neuronal cells and/or one or more tissue explants.

66. A method of measuring myelination or demyelination of one or more axons of one or a plurality of neuronal cells and/or one or a plurality of tissue explants, said method comprising:
(a) culturing one or more neuronal cells and/or one or a plurality of tissue explants on the permeable solid support of claim 1 for a time and under conditions sufficient to grow at least one axon; and
(b) positioning the insert into the adapter of an adapter comprising:
(i) a body defining a substantially flat and planar configuration with a top surface and a bottom surface;
(ii) one or plurality of planar electrodes on the top surface of the body;
(iii) a layer of insulating material; and
(iv) a circular or substantially cylindrical collar positioned on its edge around a central opening formed and extending through the body;
(c) inducing a compound action potential in the one or more neuronal cells and/or one or more tissue explants;
(d) measuring the compound action potential; and
(e) quantifying the levels of myelination of such one or more neuronal cells based on the compound action potential.
